(19) United States
(12) Patent Application Publication
(10) Pub. No.: US 2018/0374572 A1
(43) Pub. Date: Dec. 27, 2018

(54) PHYSICAL THERAPY, HEALTH, AND WELLNESS SERVICES COORDINATION SYSTEM AND METHOD

(71) Applicant: Nue Wellness LLC, Pinescrest, FL (US)

(72) Inventor: Shelley Ackerman, Pinecrest, FL (US)

(21) Appl. No.: 16/017,995

(22) Filed: Jun. 25, 2018

Related U.S. Application Data
(60) Provisional application No. 62/524,849, filed on Jun. 26, 2017.

(51) Int. Cl. G16H 40/20; G06Q 40/08; G06Q 20/40; G06Q 20/12; G06Q 10/10

(52) U.S. Cl. CPC G16H 40/20; G06Q 40/08; G06Q 10/1095; G06Q 20/127; G06Q 20/40

(57) ABSTRACT

A physical therapy, health, and wellness services coordination system is provided for coordinating physical therapy, health, and wellness services using an electronically accessible system operated on a network-connected device with a display. The physical therapy, health, and wellness services coordination system may include a dashboard component, customer module, provider module, onboarding module, service coordination component, on-demand module, matching module, commerce component, payment module, and insurance module. A method to operate a coordinating physical therapy, health, and wellness services using an electronically accessible system is also provided.

### CROSS-REFERENCE TO RELATED APPLICATION

This application claims the priority from U.S. provisional patent application Ser. No. 62/524,849 filed Jun. 26, 2017. The foregoing application is incorporated in its entirety herein by reference.

### FIELD OF THE INVENTION

The present disclosure relates to a physical therapy, health, and wellness services coordination system. More particularly, the disclosure relates to coordinating physical therapy, health, and wellness services using an electronically accessible system operated on a network-connected device with a display.

### BACKGROUND

Maintaining a healthy lifestyle may lead to increased productivity, decreased needs for medical services, and possibly greater enjoyment of life. Health and wellness services may be directed to helping potential customers maintain a healthy lifestyle. However, it may be difficult for potential health and wellness customers to locate a wellness service provider that fits the needs of the customer. Additionally, a potential health and wellness customer may be deterred from engaging in health and wellness services if he or she must take multiple steps to locate a nearby wellness service provider, determine availability, schedule a session, determine if a provider is linked with a health insurance carried by the customer and travel to the provider location. Alternatively, the customer may designate a location for the session other than the customer's physical location, such as a clinic, gym, or other location. More potential health and wellness customers may elect to forgo steps to maintain a healthy lifestyle if too many steps or hurdles are required to book health and wellness services. No known health and wellness service coordination systems exist that solve the deficiencies of the prior art.

Additionally, patients undergoing physical therapy or other customers engaging in health and wellness services may be receiving care through a dedicated facility as an in-patient. However, once the patient is discharged, they may lose access or be cut off from receiving the care they need from a provider. There currently exists no known system, platform, or method to conveniently and easily connect customers with providers for scheduled or on-demand physical therapy, health, or wellness sessions.

Furthermore, health and wellness providers typically operate or work with a business that depends on servicing customers. These health and wellness providers typically must originate customers by advertising, campaigning, memberships, and other marketing efforts. Once a health and wellness provider is contacted by a potential customer, typically multiple steps and interactions must occur before a service is booked and coordinated. For example, under current practices, incoming customers are typically required to speak with a receptionist to schedule a session, coordinate availability and/or acceptance of insurance payment options and finalize other details about a health and wellness service session. No known health and wellness coordination system exists to improve lead generation, decrease marketing costs, and streamline customer intake processes exists that solve the deficiencies of the prior art.

Therefore, a need exists to solve the deficiencies present in the prior art. What is needed is a system to help customers locate and engage sessions with physical therapy and wellness providers. What is needed is a system to help book physical therapy and wellness sessions. What is needed is a system to schedule physical therapy and wellness sessions substantially on-demand. What is needed is a system to coordinate physical therapy and wellness providers with customers using an electronically accessible system. What is needed is a system to process payment for physical therapy and wellness sessions, including filtering for insurance providers, substantially on-demand. What is needed is a system for matching physical therapy and wellness providers with potential customers based on compatibility of one or more factors, including insurance providers.

### SUMMARY

An aspect of the disclosure advantageously provides a system to help customers locate and engage sessions with physical therapy and wellness providers. An aspect of the disclosure advantageously provides a system to help book physical therapy and wellness sessions. An aspect of the disclosure advantageously provides a system to schedule physical therapy and wellness sessions substantially on-demand. An aspect of the disclosure advantageously provides a system to coordinate physical therapy and wellness providers with customers using an electronically accessible system. An aspect of the disclosure advantageously provides a system to process payment for physical therapy and wellness services, including filtering for insurance providers, substantially on-demand. An aspect of the disclosure advantageously provides a system for matching physical therapy and wellness providers with potential customers based on compatibility of one or more factors, including insurance providers.

Accordingly, the disclosure may enable a system for improving provider and customer matching. The system may include a dashboard component, a service coordination component, and a commerce component. The dashboard component may be accessed using a network-connected device with a display. The dashboard component may include a customer module, a provider module, and a mapping module. The customer module may be manipulable by the customer via the network-connected device to at least view provider information and request a session with the provider. The provider module may be manipulable by the provider via the network-connected device to at least view the session that is requested by the customer and selectively accept the session. The mapping module may be manipulable by the customer to set a location for the session and manipulable by the provider to receive the location. The service coordination component may improve sharing of information about the session between the customer and the provider. The service coordination component may include a matching module and an on-demand module. The matching module may associate the customer and the provider to coordinate the session. The matching module may apply criteria defined by the customer to filter a list of providers according to a rule. The provider may be included in the list of providers that is compliant with the rule being presented to the customer for selection to administer the session. The on-demand module may facilitate expedited scheduling of the session chronologically close to a time that the session is requested by the customer. The commerce component may improve an exchange of value from the customer requesting the session to the provider administering the session. The customer may be associated with customer information that is stored in a database. The provider may be associated with the provider information that is stored in the database. The customer information and the provider information may be stored by the database and may be accessible via the network-connected device.

In another aspect, the commerce component may include a payment module to receive at least part of the value from the customer, validate the exchange of the value that is received, and deliver the value that is validated to the provider.

In another aspect, the commerce component may further include an insurance module to compare the session that is requested, an insurance policy of the customer, and a list of acceptable insurance of the provider to determine a level of insurance compatibility. If the level of insurance compatibility is sufficient to at least partially pay for the session, the insurance module may assist with invoicing the insurance policy for the session.

In another aspect, if the level of insurance compatibility is insufficient to fully pay for the session, the insurance module may report an insurance payment deficiency to the payment module to assist with the exchange of the value defined by the insurance payment deficiency by the customer.

In another aspect, the commerce component may include a product marketplace module to sell goods. The exchange of the value for the goods that are sold may be assisted by the payment module.

In another aspect, the dashboard component may include an onboarding module to receive the customer information for the customer desiring to search for the provider for the session and store the customer information in the database, and to receive the provider information for the provider desiring to administer the session to the customer and store the provider information in the database.

In another aspect, the dashboard component may include an administrator module to at least partially adjust the criteria and to access performance data.

In another aspect, the commerce component may include a monetization module to determine and collect a subscription fee from a whitelabel provider.

According to an embodiment of this disclosure, a system may be provided for improving provider and customer matching. The system may include a dashboard component, a service coordination component, and a commerce component. The dashboard component may be accessed using a network-connected device with a display. The dashboard component may include a customer module and a provider module. The customer module may be manipulable by the customer via the network-connected device to at least view provider information and request a session with the provider. The provider module may be manipulable by the provider via the network-connected device to at least view the session that is requested by the customer and selectively accept the session. The service coordination component may improve sharing of information about the session between the customer and the provider. The service coordination component may include a matching module to associate the customer and the provider to coordinate the session. The matching module may apply criteria defined by the customer to filter a list of providers according to a rule. The provider included in the list of providers that is compliant with the rule may be presented to the customer for selection to administer the session. The session coordination component may also include an on-demand module to facilitate expedited scheduling of the session chronologically close to a time that the session is requested by the customer. The commerce component may improve an exchange of value from the customer requesting the session to the provider administering the session. The commerce component may include a payment module to receive at least part of the value from the customer, validate the exchange of the value that is received, and deliver the value that is validated to the provider. The commerce module may also include an insurance module to compare the session that is requested, an insurance policy of the customer, and a list of acceptable insurance of the provider to determine a level of insurance compatibility. If the level of insurance compatibility is sufficient to at least partially pay for the session, the insurance module may assist with invoicing the insurance policy for the session. If the level of insurance compatibility is insufficient to fully pay for the session, the insurance module may report an insurance payment deficiency to the payment module to assist with the exchange of the value defined by the insurance payment deficiency by the customer.

In another aspect, the dashboard component may include a mapping module manipulable by the customer to set a location for the session and manipulable by the provider to receive the location. The customer may be associated with customer information that is stored in a database. The provider may be associated with the provider information that is stored in the database. The customer information and the provider information stored by the database may be accessible via the network-connected device.

In another aspect, the dashboard component may include an onboarding module to receive the customer information for the customer desiring to search for the provider for the session and store the customer information in the database, and to receive the provider information for the provider desiring to administer the session to the customer and store the provider information in the database.

In another aspect, the commerce component may include a product marketplace module to sell goods. The exchange of the value for the goods that are sold may be assisted by the payment module.

In another aspect, the dashboard component may include an administrator module to at least partially adjust the criteria and to access performance data.

In another aspect, the commerce component may include a monetization module to determine and collect a subscription fee from a whitelabel provider.

According to an embodiment of this disclosure, a method may be provided for improving provider and customer matching. The method may include (a) viewing provider information and requesting a session by the customer via manipulating a customer module of a dashboard component via a network-connected device with a display. The method may include (b) viewing the session that is requested by the customer and selectively accepting the session by the provider via manipulating a provider module of the dashboard component via the network-connected device. The method may include (c) associating the customer and the provider via a matching module of a service coordinating component to coordinate the session. The operation of step (c) may further include (i) applying criteria defined by the customer to filter a list of providers according to a rule, and (ii) presenting to the customer the provider included in the list of providers that is compliant with the rule for selection to administer the session. The method may include (d) defining a location for the session. The operation of step (d) may further include (i) setting the location by the customer via a mapping component, and (ii) receiving the location by the provider via the mapping component. The method may include (e) exchanging value from the customer requesting the session to the provider administering the session via a commerce component. The operation of step (e) may further include (i) receiving at least part of the value from the customer via a payment module, (ii) validating an exchange of the value that is received, and (iii) delivering the value that is validated to the provider. The method may include (f) comparing the session that is requested, an insurance policy of the customer, and a list of acceptable insurance of the provider to determine a level of insurance compatibility via an insurance module. The operation of step (f) may further include (i) assisting with invoicing the insurance policy for the session if the level of insurance compatibility is sufficient to at least partially pay for the session, and (ii) reporting an insurance payment deficiency to the payment module to assist with the exchange of the value defined by the insurance payment deficiency by the customer if the level of insurance compatibility is insufficient to fully pay for the session.

In another embodiment, the method may further include after step (b), (g) scheduling the session chronologically close to a time that the session is requested by the customer via an on-demand module of the service coordination component. The customer may be associated with customer information that is stored in a database. The provider may be associated with the provider information that is stored in the database. The customer information and the provider information stored by the database may be accessible via the network-connected device.

In another aspect, the method may include (h) receiving the customer information via an onboarding module for the customer desiring to search for the provider for the session and storing the customer information in the database. The method may additionally include (j) receiving the provider information via the onboarding module for the provider desiring to administer the session to the customer and storing the provider information in the database.

In another aspect, the method may include (k) selling goods via a product marketplace module of the commerce component. The exchange of the value for the goods that are sold may be assisted by the payment module.

In another aspect, the method may include (l) at least partially adjusting the criteria and accessing performance data via an administrator module of the dashboard component.

In another aspect, the method may include (m) determining and collecting a subscription fee from a whitelabel provider via a monetization module of the commerce component.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

### BRIEF DESCRIPTION OF THE DRAWINGS

FIG. 1 is a block diagram view of an illustrative physical therapy, health, and wellness services coordination system, according to an embodiment of this disclosure.

FIG. 2 is a block diagram view of an illustrative computerized device, according to an embodiment of this disclosure.

FIG. 3 is a block diagram view of an illustrative networking structure, according to an embodiment of this disclosure.

FIG. 4 is a block diagram view of an illustrative customer interface structure, according to an embodiment of this disclosure.

FIG. 5 is a block diagram view of an illustrative provider interface structure, according to an embodiment of this disclosure.

FIG. 6 is a flowchart view of an illustrative operation for locating available physical therapy, health, and wellness providers, according to an embodiment of this disclosure.

FIG. 7 is a flowchart view of an illustrative operation for selecting and scheduling physical therapy, health, and wellness services generally, according to an embodiment of this disclosure.

FIG. 8 is a flowchart view of an illustrative operation for a physical therapy module, according to an embodiment of this disclosure.

FIG. 9 is a flowchart view of an illustrative operation for a massage module, according to an embodiment of this disclosure.

FIG. 10 is a flowchart view of an illustrative operation for a personal training module, according to an embodiment of this disclosure.

FIG. 11 is a flowchart view of an illustrative operation for a provider accepting a session and providing service, according to an embodiment of this disclosure.

FIG. 12 is a flowchart view of an illustrative commerce component operation, according to an embodiment of this disclosure.

FIG. 13 is a flowchart view of an illustrative insurance module operation, according to an embodiment of this disclosure.

FIG. 14 is a flowchart view of an illustrative operation for a rebooking module, according to an embodiment of this disclosure.

FIG. 15 is a flowchart view of an illustrative operation of an interface usable by a customer, according to an embodiment of this disclosure.

FIG. 16 is a flowchart view of an illustrative operation of an interface usable by a provider, according to an embodiment of this disclosure.

### DETAILED DESCRIPTION

The following disclosure is provided to describe various embodiments of a physical therapy, health, and wellness services coordination system. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

Various aspects of the present disclosure will now be described in detail, without limitation. In the following disclosure, a physical therapy, health, and wellness services coordination system will be discussed. Those of skill in the art will appreciate alternative labeling of the physical therapy, health, and wellness services coordination system as a wellness matching system, physical therapy matching system, physical therapy and wellness matching application, wellness app, matching system, app, the invention, or other similar names. Similarly, those of skill in the art will appreciate alternative labeling of the physical therapy, health, and wellness services coordination system as a wellness provider matching method, physical therapist matching method, health and wellness matching method, wellness contractor coordinating method, matching method, method, operation, the invention, or other similar names. Skilled readers should not view the inclusion of any alternative labels as limiting in any way.

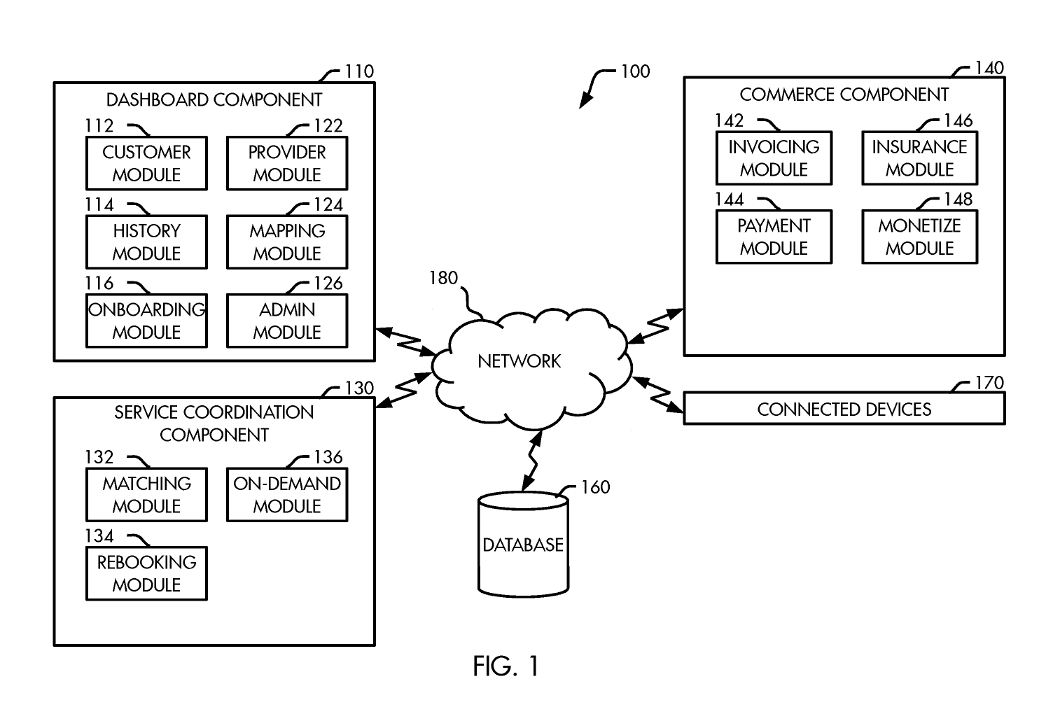

Referring now to FIGS. 1-16, the physical therapy, health, and wellness services coordination system will now be discussed in more detail. A physical therapy, health, and wellness services coordination system, an example of which is shown in block diagram 100 of FIG. 1, may include a dashboard component 110, customer module 112, onboarding module 116, provider module 122, service coordination component 130, matching module 132, rebooking module 134, on-demand module 136, commerce component 140, payment module 144, insurance module 146, and additional components that will be discussed in greater detail below. The physical therapy, health, and wellness services coordination system may operate one or more of these components interactively with other components for coordinating physical therapy, health, and wellness services using an electronically accessible system.

The disclosure generally may relate to a system for coordinating health and wellness services between users, including customers (health and wellness customers, clients, etc.) and wellness service providers (physical therapists, personal trainers, etc.), for sharing information relating to wellness and related services, facilitating providing of health and wellness services, and collecting payment. The disclosure also may include methods for the same. The system and method may coordinate customers with service providers that would provide the health and wellness services.

The disclosure may include multiple logical components operable on a computerized device with a processor and memory. Various components may be operated using a shared processor and/or memory. Alternatively, one or more components may be operated on independently managed processors and/or memory. One or more of the components may be operated collectively with additional components of this disclosure.

The components may include modules, which may provide instructions to perform desired operations discussed throughout this disclosure. Information may be stored and/or communicated using a database, which may be operatively connected over a network. The disclosure may include one or more server devices, which may connect over a network to share information. Users may interact with the components and modules of this disclosure using an interface operable on a computerized device such as a smartphone or personal computer. Customers and service providers may optionally be given targeted interfaces respective to the unique needs of a customer or a service provider. Interfaces may be provided in multiple languages, for example, English, Spanish, French, Chinese, Korean, Thai, and/or other languages.

The dashboard component will now be discussed in greater detail. FIGS. 1 and 3-13 highlight examples of the dashboard component, which may also be shown in other figures. The dashboard component 110 may help organize operation of the components and modules. The dashboard component 110 may include various modules, such as a customer module 112, provider module 122, history module 114, mapping module 124, onboarding module 116, and/or additional modules. In some embodiments, aspects of the dashboard component 110 may be provided in a “whitelabel” version, which may include customizations for a third party licensing use of the system. For example, an insurance provider may customize the appearance of the system to display their logo and apply a filter to only show providers accepted by their health insurance network.

The dashboard component 110 may optionally include an administrator module 126, which may provide access to performance data related to operation of the system by an administrator, operator of the system, or other party. Performance data accessible using the administrator module 126 could include usage history, statistics, profile information, payment details, transaction history, financial information, data transmission statistics, and other data that may relate to use of the system. An administrator may additionally adjust criteria relating to matching, rules, and other aspects that affect the operation of a system enabled by this disclosure. An administrator or other party accessing this data may run reports based on the data, derive additional information from the data, and otherwise interact with the data in a way that would be apparent to a person of skill in the art.

The customer module will now be discussed in greater detail. FIGS. 1, 3, and 6-10 highlight examples of the customer module, which may also be shown in other figures. In one embodiment, the dashboard component 110 may include a customer module 112, which may facilitate interaction with various aspects of this disclosure by a customer. The customer module 112 may include links to desirable features, such as selecting and scheduling of health and wellness services, viewing customer profile information, editing customer profile information, viewing and editing wallet and payment information, and other features that could be operated by a customer. The customer module 112 may additionally allow a customer to rate or review a provider. These ratings and reviews may be used to filter results produced by other components of this disclosure. The customer module 112 may facilitate navigation of the dashboard component 110 and other connected components.

The provider module will now be discussed in greater detail. FIGS. 1, 4, and 11 highlight examples of the provider module, which may also be shown in other figures. In one embodiment, the dashboard component 110 may include a provider module 122, which may facilitate interaction with various aspects of this disclosure by a provider. The provider module 122 may include links to desirable features, such as viewing session requests by customers, viewing provider profile information, editing provider profile information, viewing and editing accepted payment methods, viewing and editing payment accounts, and other features that could be operated by a provider. The provider module 122 may facilitate navigation of the dashboard component 110 and other connected components. A provider may receive notifications of available service request through the provider module 122. These notifications may be turned on and off using the provider module 122. The provider may optionally select visibility of the provider's location through the provider module 122.

A history module 114 may be included by the dashboard component 110. The history module 114 may provide information to a user about prior use of the dashboard component 110 and other aspects of this disclosure.

As a user interacts with the dashboard component 110, service coordination component 130, commerce component 140, or other aspects of the system, a history record may be created, updated, and/or otherwise maintained. The history record may be stored on a connected database 160. The history module 114 may retrieve data about the history record from a connected database 160 to display to a user. The information recorded and/or retrievable about historical use may be filtered according to the type of user requesting the information. For example, historical information requested by a customer, such from a customer module, may be filtered to show past sessions associated with the requesting customer, providers associated with the filtered sessions, and other information that relates to activity by the requesting customer. Alternatively, for example, historical information requested by a provider, such as from a provider module 122, may be filtered to show past sessions accepted by the provider, sessions fulfilled by the provider, customers for which services were provided by the provider, payments received, and other information that relates to activity by the requesting provider.

A mapping module 124 may additionally be included by the dashboard component 110 to facilitate visualization of prospective providers and customers on a map. The mapping module 124 may use information received by a geolocation component of an electronic device to indicate a position of a user on the map, such as by using GPS, triangulation, or another location technique that would be appreciated by a person of skill in the art. The mapping component 124 may help customers to view providers within an acceptable vicinity that are available to provide health and wellness services.

Additionally, the mapping module 124 may assist with the rendering of coordinated health and wellness services. For example, the mapping module 124 may provide information to matched customers and providers to help with providing the health and wellness services. The mapping module 124 may display to a provider a location of a matched customer for which health and wellness services are to be provided. For example, information from a GPS sensor in a customer smartphone may be shared with a matched provider to facilitate a meeting.

Additional aspects of the mapping module 124 and other components may assist with scheduling a session at a hotel or other temporary location. For example, while booking a session to be administered at a hotel room, the customer may be asked for the name of the hotel, room number, gate or door code, whether the customer has the necessary equipment such as a table or gym equipment, fitness level of the customer, pets, need to climb stairs, other pertinent medical information, and/or other details that may relate to the administering of the session by the provider.

In one example, the mapping module 124 may additionally display to a customer a location of a matched provider to inform the customer of an estimated time of arrival. The matched provider may interact with an interface to indicate that the provider is beginning the route to the customer. The customer may be given substantially live updates of the provider's location. The provider may then interact with the interface to indicate that the provider has arrived at the location. Once the provider and customer meet in person, the provider may interact with the interface to indicate that the route has completed. Additionally, a matched customer and provider may interact with the mapping module 124 to coordinate a meeting location to begin the health and wellness session.

The onboarding module will now be discussed in greater detail. FIG. 1 highlights examples of the onboarding module, which may also be shown in other figures. An onboarding module 116 may be provided to receive new users, including customers, wellness providers, or other parties. For example, the onboarding module may receive customer information relating to a new customer and/or provider information relating to a new provider. The customer information and the provider information may be stored on the database, which may be connected via a network. The onboarding module may additionally receive subsequent customer information and/or provider information to update a profile relating to a customer and/or provider. Information gathered for a new user may be stored in the database 160 and may be used to populate a user profile. The onboarding module 116 may assist with gathering and verifying of information that can be used by the system to coordinate customers with wellness service providers.

The onboarding module 116 may facilitate registration of new users. In one embodiment, the onboarding module 116 may advantageously facilitate registration of new customers and providers. For example, the onboarding module 116 may request information about a new customer, including a name, address, preferred services, special needs, medical requirements, accessibility accommodations, insurance plans, home location, and other information. In another example, the onboarding module 116 may request information about a new provider, including name, business information, address, provided services, accommodations offered, insurance plans accepted, geographic service borders, and other information.

The dashboard component 110 may optionally include an educational module, through which providers and other parties may share educational and/or other information to customers. Providers and customers may interact with each other through the educational module. For example, a provider may share educational information regarding personal training for strength building. By sharing this information, providers may advantageously build credibility for their areas of practice offered through the system. In an additional embodiment, a provider may charge an access fee to view the information shared through the educational module.

In some embodiments, additional modules may be included by the dashboard component 100. For example, an appointment module may be provided for viewing and interacting with appointments. In another example, a profile module may be provided for viewing and editing profile information. The profile module may be customizable to work with a customer module and/or provider module. Additionally, a wallet module may be included for storing payment information. Skilled artisans will appreciate other modules that may be included by the dashboard component after having the benefit of this disclosure.

The service coordination component will now be discussed in greater detail. FIGS. 1, 7, 11, and 13-14 highlight examples of the service coordination component, which may also be shown in other figures. The service coordination component 130 may assist with matching a customer to a wellness service provider. The service coordination component 130 may include modules to help customers filter service providers by practice area (physical therapy, personal trainer, etc.), specialty (geriatrics, pediatrics, etc.), location, availability, accepted payment, guest access, insurance acceptance, and other criteria. Illustrative modules included by the service coordination component 130 include a matching module 132, rebooking module 134, and an on-demand module 136, without limitation. Customers may request a preferred time for a session, which may be reviewed and approved/denied by a service provider. Alternatively, customers may request an “on-demand” session with an available service provider similar to or matching the customer-provided criteria.

The service coordination component 130 may include various specialization aspects, which may be modified to focus on coordinating information relating to a specified service. For example, the service coordination component 130 may include a physical therapy module, massage module, personal training module, and/or other modules. In one example, the physical therapy module may help coordinate aspects unique to the module, such as insurance coverage. In another example, the massage module may help coordinate unique aspects, such as guest access for the wellness service. Other unique aspects for each includable module may also be defined to help coordination between customers and service providers.

The matching module will now be discussed in greater detail. FIGS. 1 and 6-11 highlight examples of the matching module, which may also be shown in other figures. The matching module 132 may associate a customer with a provider to coordinate a wellness session. The matching module 132 may receive coordination requests from a customer desiring health and wellness services. The matching module 132 may additionally receive criteria for the request made by the customer.

The criteria defined by the customer may be analyzed to create rules for selecting a provider to be matched. Available providers may be filtered according to the rules, enhancing compliance with the criteria. A list of providers compliant with the rules, wherein one or more of the rules may correspond with the criteria or that may approximate the criteria, may be presented to the customer for selection. In one embodiment, a provider at least partially compliant with the rules may be selected to be matched with the requesting customer.

Once a customer and a provider are matched, the matching module 132 may provide contact assistance for the customer and provider to communicate. Examples of providable contact information may include, without limitation, phone number, text interface, mobile phone link, integrated messenger, and other communication interfaces that would be appreciated by a person of skill in the art after having the benefit of this disclosure.

The customer and/or provider may be given an opportunity to approve the matched party prior to finalizing coordination of the wellness session. Upon approval of the suggested match by the customer and/or provider, the session may be coordinated and finalized between the matched customer and provider.

The on-demand module will now be discussed in greater detail. FIGS. 1 and 6-11 highlight examples of the on-demand module, which may also be shown in other figures. As mentioned above, an on-demand module 136 to assist with expedited scheduling features may be included that may connect a customer with a wellness service provider for a current or upcoming schedule opening. For example, the customer may select to schedule a session “on-demand.” Available service providers may be searched that are compliant with definable criteria. For example, possible criteria may define wellness service providers in an accessible geographic area with openings in their schedules. One or more wellness service providers substantially matching the search criteria may then be recommended to the customer. The on-demand module 136 may then assist with making a session in a relatively soon time slot. In one example, the on-demand module 136 may select a wellness service provider with a nearly immediate opening in his or her schedule and located near the requesting customer, which may minimize the time required between the request and start of the health and wellness services. Examples of chronologically close times could be 5 minutes, 15 minutes, 30 minutes, 45 minutes, 1 hour, 1.5 hours, 2 hours, 3 hours, same-day, or another increment of time that would be apparent to a skilled artisan after having the benefit of this disclosure, without limitation.

In one embodiment of the on-demand module 136, when a customer makes a request for a service, a notification may be sent to providers matching the criteria of the requested service. The criteria may include type of health or wellness service provided, geographic proximity, accepted payment methods, accepted health care insurance policies, and other criteria. The request may be accepted on a first-come-first-serve basis, with the first provider to accept the service request to be matched with the requesting customer. If the match cannot be completed, a backup provider may be selected or alternatively the notification may be resent to providers matching the criteria.

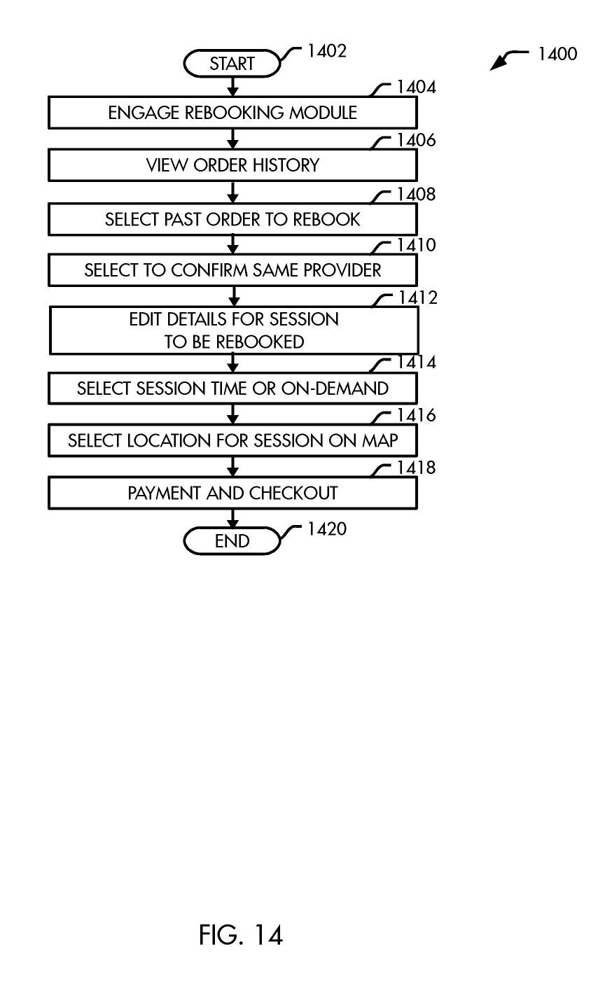

The rebooking module will now be discussed in greater detail. FIGS. 1 and 14 highlight examples of the rebooking module, which may also be shown in other figures. The rebooking module 134 may be optional. The rebooking module 134 may additionally assist with scheduling of follow-up sessions, which may help with retention of customers by wellness providers and scheduling consistency for customers. For example, a customer may select a previous session to rebook with the same wellness service provider. Optionally, some details may be modifiable when rebooking a session, such as session length. In one example, a customer may select between scheduled sessions or on-demand sessions at each rebooking cycle.

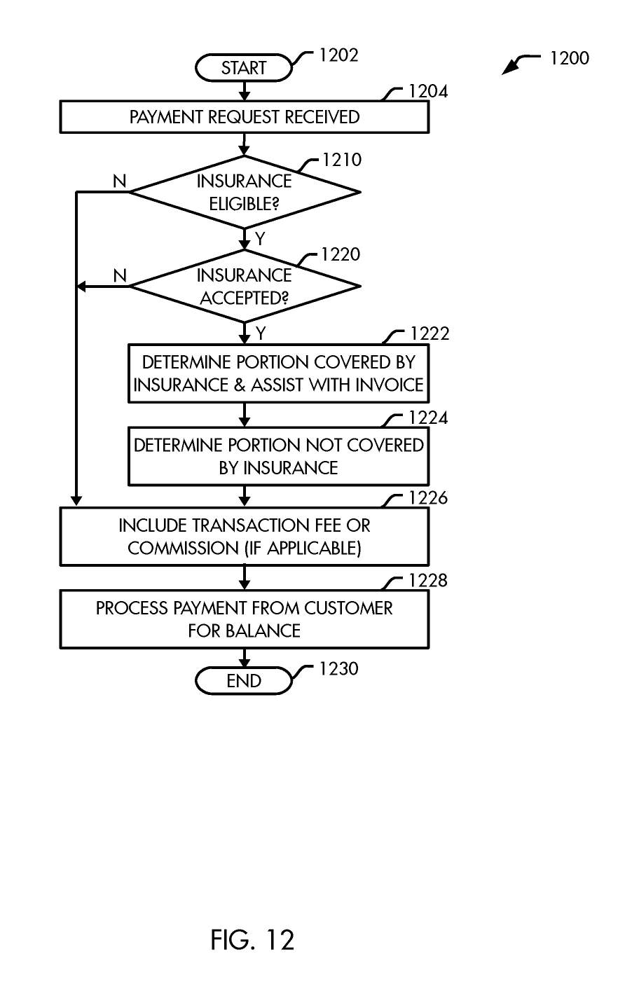

The commerce component will now be discussed in greater detail. FIGS. 1 and 12 highlight examples of the commerce component, which may also be shown in other figures. The commerce component may assist with the exchange of value or compensation from a customer requesting the session and the provider administering the session. The commerce component 140 may provide pricing, generate invoices, receive payments, calculate commissions, determine fees, and otherwise assist with the commercial aspects of providing health and wellness services. The commerce component 140 may additionally assess and collect membership fees to be included as a provider, receive leads, and be given an opportunity to accept service requests from customers. The commerce component 140 may work with the other components of the system to facilitate booking sessions for health and wellness services. Optionally, payment information can be saved for future transactions.

The commerce component 140 may include one or more modules to assist with processing commercial transactions. For example, the commerce component 140 may include an invoicing module 142, insurance module 146, payment module 144, monetization module 148, and/or additional modules that would be appreciated by those of skill in the art after having the benefit of this disclosure.

In some embodiments, the commerce component 140 may include or assist with a merchandise storefront. For example, and operator of the system may offer goods for sale to customers and/or providers, such as trigger point balls, lotion, and other products. In another example, providers may offer goods for sale via the merchandise storefront. A commission may be collected from sales by a provider using this system. Skilled artisans will appreciate other features includable by a merchandise storefront after having the benefit of this disclosure. The merchandise storefront may additionally be operable with the product marketplace module, which will be discussed in greater detail below.

The invoicing module will now be discussed in greater detail. FIG. 1 highlights examples of the invoicing module, which may also be shown in other figures. The invoicing module 142 may assist with creating invoices for a coordinated wellness service. For example, if a customer selects and schedules a 60-minute personal trainer session at $99/hour, the invoicing module 142 may generate the invoice payable by the customer. The invoice may also be accessed by the provider to assist with maintaining proper accounting records. In some embodiments, multiple parties may be invoiced for a coordinated health and wellness service session. In some embodiments, the invoicing module 142 may assist a provider with generating invoices to submit to insurance for payment external to a system enabled by this disclosure, as will be appreciated by those of skill in the art after having the benefit of this disclosure. Insurance that can be invoiced internally, externally, or other otherwise via the system may include private health insurance, employer-provided health insurance, marketplace health insurance, public health insurance, state health insurance, Medicare, Medicaid, or virtually any other health insurance or other insurance organization.

For some providers, such as providers not accepting payment through the system, the invoicing module 142 may invoice the provider for a subscription fee, such as a membership fee, to access the system of this disclosure. Providers may be onboarded with a membership having a definable term, for example, 3 months, 6 months, 9 months, or another term period. In some embodiments, invoicing may be provided as a whitelabel service. For example, a contractor of a system enabled by this disclosure may apply their contractor branding to the invoices and other aspects of the system.

The payment module will now be discussed in greater detail. FIG. 1 highlights examples of the payment module, which may also be shown in other figures. The payment module 144 may provide a tool for customers to submit payments for coordinated health and wellness services. In one embodiment, the payment module 144 may include an option to authorize the purchase for a selected wellness service session. In another embodiment, the payment module 144 may substantially automatically process payment for a selected wellness service session.

In one example, the payment module may receive a value from a customer, which may include a complete or partial payment for the session administered by, and/or products sold by, the provider. An example of a partial payment may include a co-pay accompanying an insurance-billable session, without limitation. The payment module may validate the value received by the customer, for example, via a payment gateway, currency exchange, bank, or other transaction validation technique that would be apparent to a skilled artisan after having the benefit of this disclosure. The validated portion of the value may then be delivered to the provider, for example, as at least partial payment for a session.

Skilled artisans will appreciate additional embodiments by which payments may be authorized and processed. In some embodiments, packages may be purchased in advance, such as a three-hour massage package. The time in a purchased package may be configured to be used at once, split between multiple sessions, giftable, or otherwise transferrable.

The insurance module will now be discussed in greater detail. FIG. 1 highlights examples of the insurance module, which may also be shown in other figures. In some embodiments, the insurance module 146 may be an optional feature. In other embodiments, the insurance module 146 may be an important aspect to a system enabled by this disclosure, such as for a session including insurance-eligible services. The insurance module 146 may assist with comparing an insurance policy of a customer and list of acceptable insurance of a provider to determine a level of insurance compatibility, which may consider factors such as whether a service performed in a session is insurance-eligible, whether products or goods used to assist with the session are insurance-eligible, a level of coverage that applies to services performed in the session, itemization, provider, hospital affiliation if applicable, licensure, and other factors that would be appreciated by a person of skill in the art after having the benefit of this disclosure, without limitation. The insurance module 146 may additionally assist with billing insurance providers for covered sessions internally, externally, or otherwise. The insurance module 146 may compare the requested health and/or wellness service, accepted health and wellness services by a customer insurance policy, acceptance of insurance providers by a wellness service provider, level of insurance compatibility between the insurance accepted by the provider and the insurance policy of the customer, and/or other factors to determine an amount billable to insurance within the level of insurance compatibility, if any. If a service performed in a session is not insurance-eligible, the level of compatibility may be approximately null, which may essentially bypass the determination and/or assistance with billing an insurance provider.

In some embodiments, for example where the level of insurance compatibility does not permit full payment by the insurance provider for the session, an insurance payment deficiency may be defined. The insurance module 146 may optionally cooperate with the invoicing module for billing an insurance provider the permitted amount. The remaining amount may be billed to the customer directly, for example, via the payment module 144. Alternatively, the insurance module 146 may assist with gathering information from a customer to assist the provider in requesting payment from an insurance provider outside of the system.

The monetization module will now be discussed in greater detail. The monetization module 148 may determine a subscription fee payable to an operator of a system, for example, a whitelabel provider. For the purposes of this disclosure, a whitelabel provider is defined as an operator of a system enabled by this disclosure other than the developer of the system. A whitelabel provider may license use of the system and/or may apply their branding to the system. In one embodiment, the monetization module 148 may calculate a commission for each coordinated wellness service session. In another embodiment, the monetization module 148 may collect a subscription fee from providers wishing to be selected by the service coordination component. The determination by the monetization module may be variable, dynamic, fixed, application of a set amount, or otherwise determined as will be appreciate by those of skill in the art. Additional rules may be defined by which payments made through the commerce component may be monetized, which would be apparent to a person of skill in the art after having the benefit of this disclosure.

Customers, providers, and/or other parties may interact with the components via connected devices 170. Examples of connected devices 170 may include smartphones, mobile computing platforms, personal computers, and other computerized devices. One or more of the components and/or modules may communicate over a network 180. For example, the dashboard component 110 may be accessed over a network 180 by a customer using a connected device 170. The dashboard component 110 may communicate with a database 160 to provide information to the customer about his or her account, coordinate a wellness service session, or perform another operation provided by this disclosure. One or more of the components, modules, databases, connected devices, and/or other aspects may be operated on a shared physical computerized device.

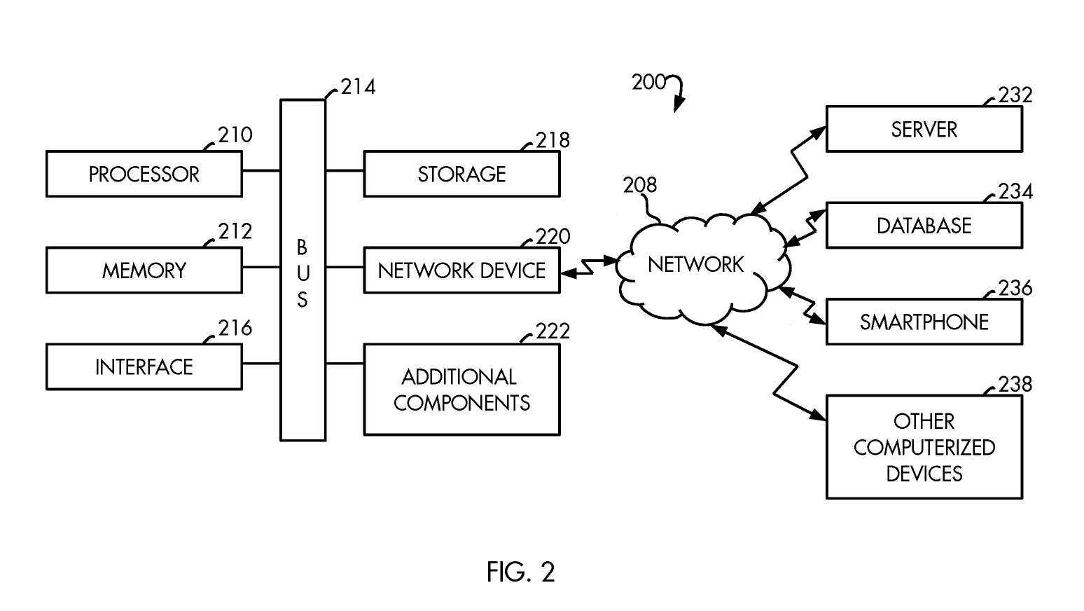

Referring now to FIG. 2, an illustrative computerized device will be discussed, without limitation. Various aspects and functions described in accord with the present disclosure may be implemented as hardware or software on one or more illustrative computerized devices 200 or other computerized devices. There are many examples of illustrative computerized devices 200 currently in use that may be suitable for implementing various aspects of the present disclosure. Some examples include, among others, network appliances, personal computers, workstations, mainframes, networked clients, servers, media servers, application servers, database servers and web servers. Other examples of illustrative computerized devices 200 may include mobile computing devices, cellular phones, smartphones, tablets, video game devices, personal digital assistants, network equipment, devices involved in commerce such as point of sale equipment and systems, such as handheld scanners, magnetic stripe readers, bar code scanners and their associated illustrative computerized device 200, among others. Additionally, aspects in accord with the present disclosure may be located on a single illustrative computerized device 200 or may be distributed among one or more illustrative computerized devices 200 connected to one or more communication networks.

For example, various aspects and functions may be distributed among one or more illustrative computerized devices 200 configured to provide a service to one or more client computers, or to perform an overall task as part of a distributed system. Additionally, aspects may be performed on a client-server or multi-tier system that includes components distributed among one or more server systems that perform various functions. Thus, the disclosure is not limited to executing on any particular system or group of systems. Further, aspects may be implemented in software, hardware or firmware, or any combination thereof. Thus, aspects in accord with the present disclosure may be implemented within methods, acts, systems, system elements and components using a variety of hardware and software configurations, and the disclosure is not limited to any particular distributed architecture, network, or communication protocol.

FIG. 2 shows a block diagram of an illustrative computerized device 200, in which various aspects and functions in accord with the present disclosure may be practiced. The illustrative computerized device 200 may include one or more illustrative computerized devices 200. The illustrative computerized devices 200 included by the illustrative computerized device may be interconnected by, and may exchange data through, a communication network 208. Data may be communicated via the illustrative computerized device using a wireless and/or wired network connection.

Network 208 may include any communication network through which illustrative computerized devices 200 may exchange data. To exchange data via network 208, systems and/or components of the illustrative computerized device 200 and the network 208 may use various methods, protocols and standards including, among others, Ethernet, Wi-Fi, Bluetooth, TCP/IP, UDP, HTTP, FTP, SNMP, SMS, MMS, SS7, JSON, XML, REST, SOAP, RMI, DCOM, and/or Web Services, without limitation. To ensure data transfer is secure, the systems and/or modules of the illustrative computerized device 200 may transmit data via the network 208 using a variety of security measures including TSL, SSL, or VPN, among other security techniques. The illustrative computerized device 200 may include any number of illustrative computerized devices 200 and/or components, which may be networked using virtually any medium and communication protocol or combination of protocols.

Various aspects and functions in accord with the present disclosure may be implemented as specialized hardware or software executing in one or more illustrative computerized devices 200, including an illustrative computerized device 200 shown in FIG. 2. As depicted, the illustrative computerized device 200 may include a processor 210, memory 212, a bus 214 or other internal communication system, an input/output (I/O) interface 216, a storage system 218, and/or a network communication device 220. Additional devices 222 may be selectively connected to the computerized device via the bus 214. Processor 210, which may include one or more microprocessors or other types of controllers, can perform a series of instructions that result in manipulated data. Processor 210 may be a commercially available processor such as an ARM, x86, Intel Core, Intel Pentium, Motorola PowerPC, SGI MIPS, Sun UltraSPARC, or Hewlett-Packard PA-RISC processor, but may be any type of processor or controller as many other processors and controllers are available. As shown, processor 210 may be connected to other system elements, including a memory 212, by bus 214.

The illustrative computerized device 200 may also include a network communication device 220. The network communication device 220 may receive data from other components of the computerized device to be communicated with servers 232, databases 234, smart phones 236, and/or other computerized devices 238 via a network 208. The communication of data may optionally be performed wirelessly. More specifically, without limitation, the network communication device 220 may communicate and relay information from one or more components of the illustrative computerized device 200, or other devices and/or components connected to the computerized device 200, to additional connected devices 232, 234, 236, and/or 238. Connected devices are intended to include, without limitation, data servers, additional computerized devices, mobile computing devices, smart phones, tablet computers, and other electronic devices that may communicate digitally with another device. In one example, the illustrative computerized device 200 may be used as a server to analyze and communicate data between connected devices.

The illustrative computerized device 200 may communicate with one or more connected devices via a communications network 208. The computerized device 200 may communicate over the network 208 by using its network communication device 220. More specifically, the network communication device 220 of the computerized device 200 may communicate with the network communication devices or network controllers of the connected devices. The network 208 may be, for example, the internet. As another example, the network 208 may be a WLAN. However, skilled artisans will appreciate additional networks to be included within the scope of this disclosure, such as intranets, local area networks, wide area networks, peer-to-peer networks, and various other network formats. Additionally, the illustrative computerized device 200 and/or connected devices 232, 234, 236, and/or 238 may communicate over the network 208 via a wired, wireless, or other connection, without limitation.

Memory 212 may be used for storing programs and/or data during operation of the illustrative computerized device 200. Thus, memory 212 may be a relatively high performance, volatile, random access memory such as a dynamic random-access memory (DRAM) or static memory (SRAM). However, memory 212 may include any device for storing data, such as a disk drive or other non-volatile storage device. Various embodiments in accord with the present disclosure can organize memory 212 into particularized and, in some cases, unique structures to perform the aspects and functions of this disclosure.

Components of illustrative computerized device 200 may be coupled by an interconnection element such as bus 214. Bus 214 may include one or more physical busses (for example, busses between components that are integrated within a same machine) but may include any communication coupling between system elements including specialized or standard computing bus technologies such as USB, Thunderbolt, SATA, FireWire, IDE, SCSI, PCI and InfiniBand. Thus, bus 214 may enable communications (for example, data and instructions) to be exchanged between system components of the illustrative computerized device 200.

The illustrative computerized device 200 also may include one or more interface devices 216 such as input devices, output devices and combination input/output devices. Interface devices 216 may receive input or provide output. More particularly, output devices may render information for external presentation. Input devices may accept information from external sources. Examples of interface devices include, among others, keyboards, bar code scanners, mouse devices, trackballs, magnetic strip readers, microphones, touch screens, printing devices, display screens, speakers, network interface cards, etc. The interface devices 216 allow the illustrative computerized device 200 to exchange information and communicate with external entities, such as users and other systems.

Storage system 218 may include a computer readable and writeable nonvolatile storage medium in which instructions can be stored that define a program to be executed by the processor. Storage system 218 also may include information that is recorded, on or in, the medium, and this information may be processed by the program. More specifically, the information may be stored in one or more data structures specifically configured to conserve storage space or increase data exchange performance. The instructions may be persistently stored as encoded bits or signals, and the instructions may cause a processor to perform any of the functions described by the encoded bits or signals. The medium may, for example, be optical disk, magnetic disk or flash memory, among others. In operation, processor 210 or some other controller may cause data to be read from the nonvolatile recording medium into another memory, such as the memory 212, that allows for faster access to the information by the processor than does the storage medium included in the storage system 218. The memory may be located in storage system 218 or in memory 212. Processor 210 may manipulate the data within memory 212, and then copy the data to the medium associated with the storage system 218 after processing is completed. A variety of components may manage data movement between the medium and integrated circuit memory element and does not limit the disclosure. Further, the disclosure is not limited to a particular memory system or storage system.

Although the above described illustrative computerized device is shown by way of example as one type of illustrative computerized device upon which various aspects and functions in accord with the present disclosure may be practiced, aspects of the disclosure are not limited to being implemented on the illustrative computerized device 200 as shown in FIG. 2. Various aspects and functions in accord with the present disclosure may be practiced on one or more computers having one or more components than that shown in FIG. 2. For instance, the illustrative computerized device 200 may include specially-programmed, special-purpose hardware, such as for example, an application-specific integrated circuit (ASIC) tailored to perform a particular operation disclosed in this example. While another embodiment may perform essentially the same function using several general-purpose computing devices running Windows, Linux, Unix, Android, iOS, MAC OS X, or other operating systems on the aforementioned processors and/or specialized computing devices running proprietary hardware and operating systems.

The illustrative computerized device 200 may include an operating system that manages at least a portion of the hardware elements included in illustrative computerized device 200. A processor or controller, such as processor 210, may execute an operating system which may be, among others, an operating system, one of the above mentioned operating systems, one of many Linux-based operating system distributions, a UNIX operating system, or another operating system that would be apparent to skilled artisans. Many other operating systems may be used, and embodiments are not limited to any particular operating system.

The processor and operating system may work together define a computing platform for which application programs in high-level programming languages may be written. These component applications may be executable, intermediate (for example, C# or JAVA bytecode) or interpreted code which communicate over a communication network (for example, the Internet) using a communication protocol (for example, TCP/IP). Similarly, aspects in accord with the present disclosure may be implemented using an object-oriented programming language, such as JAVA, C, C++, C#, Python, PHP, Visual Basic .NET, JavaScript, Perl, Ruby, Delphi/Object Pascal, Visual Basic, Objective-C, Swift, MATLAB, PL/SQL, OpenEdge ABL, R, Fortran or other languages that would be apparent to skilled artisans. Other object-oriented programming languages may also be used. Alternatively, assembly, procedural, scripting, or logical programming languages may be used.

Additionally, various aspects and functions in accord with the present disclosure may be implemented in a non-programmed environment (for example, documents created in HTML5, HTML, XML, CSS, JavaScript, or other format that, when viewed in a window of a browser program, render aspects of a graphical-user interface or perform other functions). Further, various embodiments in accord with the present disclosure may be implemented as programmed or non-programmed elements, or any combination thereof. For example, a web page may be implemented using HTML while a data object called from within the web page may be written in C++. Thus, the disclosure is not limited to a specific programming language and any suitable programming language could also be used.

An illustrative computerized device included within an embodiment may perform functions outside the scope of the disclosure. For instance, aspects of the system may be implemented using an existing commercial product, such as, for example, Database Management Systems such as an SQL Server available from Microsoft of Redmond, Wash., Oracle Database or MySQL from Oracle of Redwood City, Calif., or integration software such as WebSphere middleware from IBM of Armonk, N.Y.

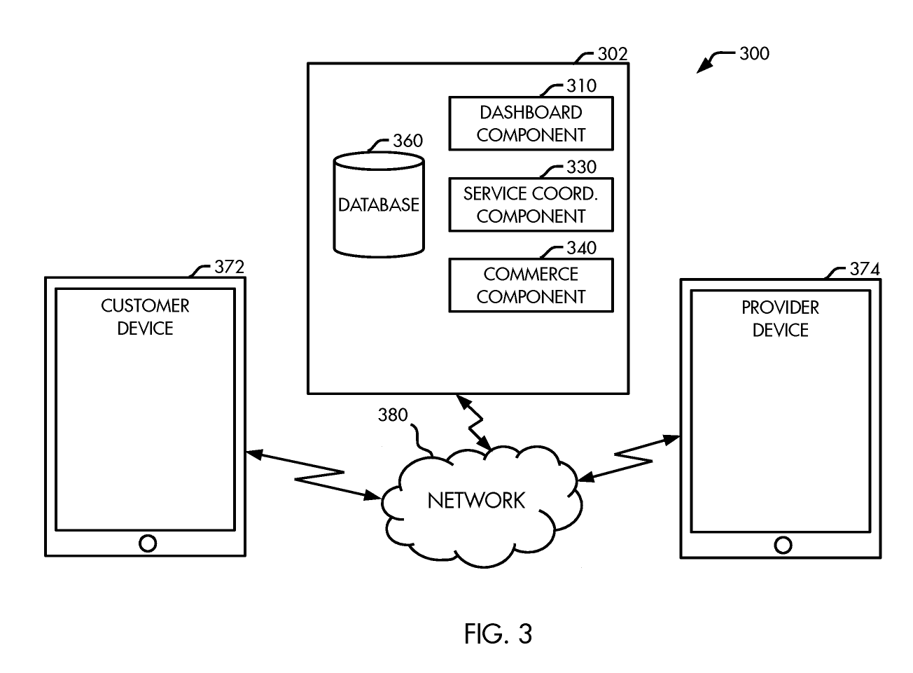

Referring now to block diagram 300 of FIG. 3, an illustrative network communication structure will be discussed, without limitation. The illustrative network communication structure may include one or more computerized devices 372, 374 that may communicate with the components, database 360, and other aspects of this disclosure over a network 380. The computerized devices may include one or more customer device 372 and one or more provider device 374. The components may include, for example, the dashboard component 310, service coordination component 330, and commerce component 340. In one embodiment, the components 310, 330, 340 and the database 360 may be hosted by a server 302 communicable with the computerized devices 372, 374 over a network 380. In another embodiment, the components may be operated using a first server and the database 360 may be operated using a second server, which may be communicably connected over a network. Skilled artisans will appreciate additional embodiments in which various components, and even modules associated with each component, may be operated on multiple servers connected over a network.

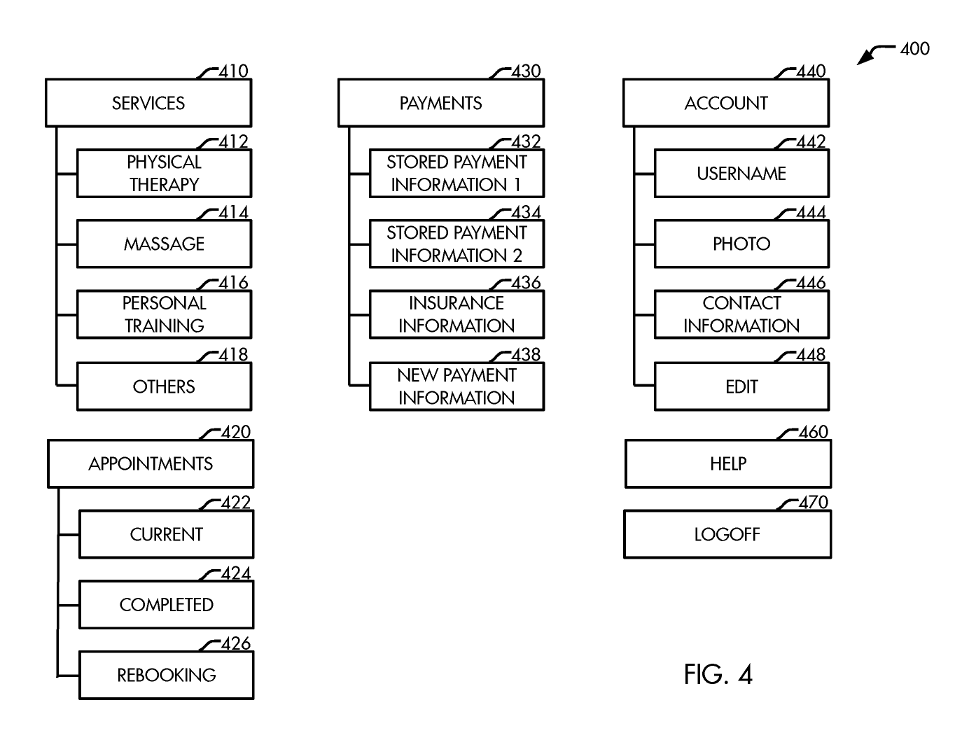

Referring now to block diagram 400 of FIG. 4, an illustrative organizational structure of an interface viewable by a customer will be discussed. This illustrative interface view may be provided by the dashboard component with assistance from the customer module. The customer interface may be access via a network-connected device, which may include a display and input device, for example, a touchscreen.

The customer interface may include a branch of features related to services offered to the customer. For example, the services branch 410 may provide a customer with navigation to search and coordinate health and wellness services relating to physical therapy 412, massage 414, personal training 416, and other health and wellness services 418. Skilled artisans will appreciate that virtually any additional services, including additional health and wellness services 418, may be accessible to a customer through the services branch 410 of the customer interface.

The customer interface may include a branch of features related to sessions. The appointment branch 420 may allow the user to view currently scheduled sessions 422. If the customer recently requested an on-demand wellness session, the appointment branch may provide the customer information about the status of the request, such as provider location and estimated time of arrival. The information may additionally provide the location of a provider or session on a map with assistance from the mapping module.

The customer may interact with the history module to view prior and completed wellness service sessions 424. Prior sessions may also be viewable on a map with the assistance of the mapping module. Through the appointments branch of the customer interface, customers may elect to rebook a prior session 426 with the assistance of the rebooking module.

The customer interface may include a branch of features related to payments. The payment branch 430 may assist customers with managing various stored payment methods. For example, the payment branch 430 may provide customers with access to manage a first stored payment information 432, a second stored payment information 434, insurance payment information 436, and other storable payment information 438. Additionally, the payments branch 430 may assist the customer with defining new payment methods.

The customer interface may include a branch of features related to account management 440. For example, a customer may view his or her username 442, password, profile photograph 444, contact information 446, and other profile information associable with the customer. An edit feature 448 may be provided to the customer to edit one or more fields of the stored profile information.

The customer interface may additionally include a help aspect 460, which may direct the customer to information about how to access, operate, and otherwise interact with the interface. The customer interface may additionally include a logoff feature 470 to logoff and/or exit the interface and components of this disclosure.

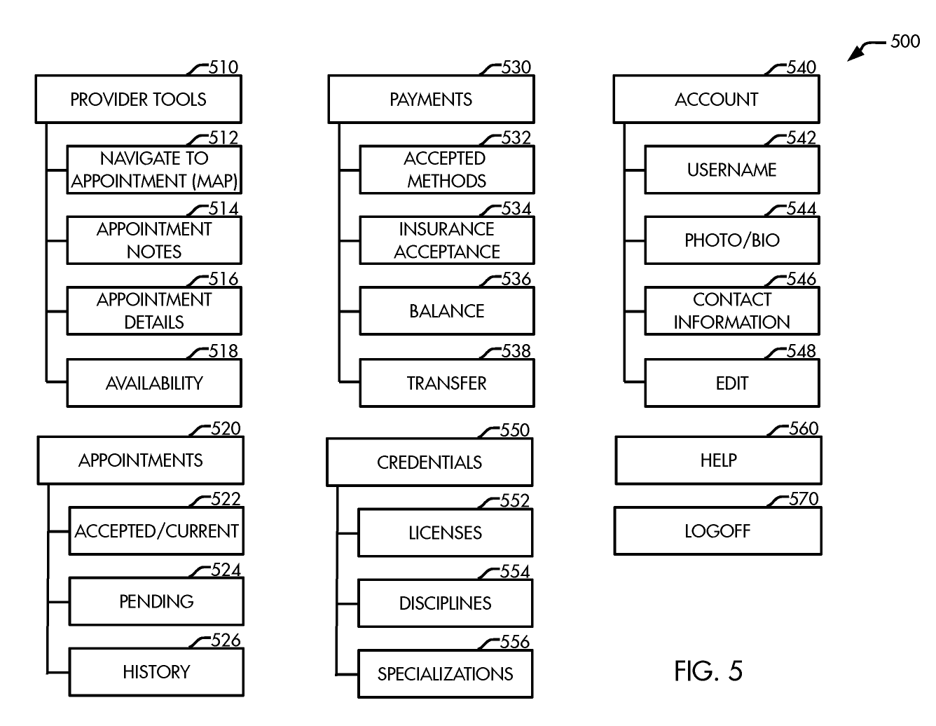

Referring now to block diagram 500 of FIG. 5, an illustrative organizational structure of an interface viewable by a provider will be discussed. This illustrative interface view may be provided by the dashboard component with assistance from the provider module. The provider interface may be access via a network-connected device, which may include a display and input device, for example, a touchscreen.

The provider interface may include a branch of features related to provider tools. For example, a provider may interact with a tool of the provider tools branch 510 to navigate to a session 512. These navigation tools may show a path to the session on a map, which may operate with the assistance of the mapping module. The provider tools may additionally allow the provider to create and view session notes 514. In some examples, session notes may be created by a customer requesting the session. The provider may also access provider tools to view session details 516. Furthermore, the provider may be given provider tools to set his or her availability to accept new sessions 518. In one example, the provider may engage an availability tool to indicate whether he or she is available to accept on-demand sessions.

The provider interface may include a branch of features related to sessions. The appointment branch 520 may allow the user to view currently scheduled sessions 522. The viewable currently scheduled sessions may include sessions for health and wellness services that have been accepted by the provider 522. The appointment branch 520 may additionally give the provider information about sessions pending acceptance 524. If the provider recently accepted to provide an on-demand wellness session, the appointment branch may provide information about the status of the request, such as customer location and estimated time of arrival. The information may additionally provide the location of a customer or session on a map with assistance from the mapping module.

The provider may interact with the history module to view prior and completed wellness service sessions 526. Prior sessions may also be viewable on a map with the assistance of the mapping module. Through the appointments branch of the provider interface, providers may view requests to rebook a prior session by a customer with the assistance of the rebooking module.

The provider interface may include a branch of features related to payments. The payment branch 530 may assist providers with managing various accepted payment credentials and associated information. For example, the payment branch 530 may provide providers with access to manage information for an accepted credit card payment 532, a second payment credential information, insurance payment information 534, and other storable payment information. Additionally, the payments branch 530 may assist the provider with defining new accepted payment methods. A provider may additionally view his or her balance accrued from providing health and wellness services 536. The provider may request a transfer of funds from or to his or bank account from the available balance using transfer aspects 538 of the payment branch 530 of the provider interface.

The provider interface may include a branch of features related to account management 540. For example, a provider may view his or her username 542, password, profile photograph 544, provider information 546, and other profile information associable with the provider. An edit feature 548 may be provided to the provider to edit one or more fields of the stored profile information.

The provider interface may include a branch of features related to credentials 550. The provider may have access to sign up for or request membership. If membership sign up is completed, and if an optional step of membership approval is completed, the prover may be given access to the provider interface.

In one example, through the provider interface, a provider may list licenses to provide various types of health and wellness services 552. The listed licenses may be confirmed prior to being listed publicly to prospective customers. The credentials branch of the provider interface may additionally include a listing of disciplines in which the provider offers health and wellness services 554. Examples of disciplines may include physical therapy, massage, personal training, and other health and wellness services that would be appreciated by a person of skill in the art after having the benefit of this disclosure.

Additionally, the credentials branch may include specializations to further inform prospective customers of the health and wellness services provided by the provider 556. In one example, a provider providing health and wellness services in the personal training discipline may list specializations such as weight loss, muscle gain, sport specific training, injury rehabilitation, core/flexibility training, endurance training, plyometrics training, explosive performance, pre/post-natal training, experience in training 55+, bootcamp group training, and other specializations. In another example, a provider providing health and wellness services in the massage discipline may list specializations such as Swedish, deep tissue, sports, pre-natal, at-work, and other specializations. In an additional example, a provider providing health and wellness services in the physical therapy discipline may list specializations such as geriatrics, pediatrics, sports, orthopedic, neurologic, women's health, and/or other specializations.

The provider interface may additionally include a help aspect 560, which may direct the provider to information about how to access, operate, and otherwise interact with the interface. The provider interface may additionally include a logoff feature 570 to logoff and/or exit the interface and components of this disclosure.

In one embodiment, a product marketplace module may be included to assist with the sales of goods and products that may be related to the physical therapy, health, and/or wellness services offered by a provider. Products listed through the product marketplace module may be organized by categories, subcategories, tags, specialty, discipline, insurance coverage, and otherwise. Products may be filtered, sorted, ordered, and otherwise organized. For example, products may be sorted by most relevant, new and popular, by price, highest rating, and other criteria. Some products may be featured in the marketplace. Additionally, some products may offer customization options, for example, material, size, color, or other options. Payment for goods and/or products sold through the product marketplace module may be at least partially assisted by the payment module 144 and/or insurance module 146.

A rating module may be included to for the parties to a session to rate one another. For example, a customer may access an interface through the customer module to rate a provider that just administered a session. The rating may include number of stars, comments, feedback, detailed breakdown, and other rating aspects that would be appreciated by a person of skill in the art. Additionally, a provider may access an interface through the provider module to rate a customer just serviced during a session. The rating may include number of stars, comments, feedback, detailed breakdown, and other rating aspects that would be appreciated by a person of skill in the art. Ratings and comments may be moderated, for example, to avoid negative impact on an otherwise good provider by a customer that consistently leaves negative feedback.

A customer and/or provider may be given access to the product marketplace component through an interface to purchase products that may assist with the provider administering a physical therapy session. The products may be selectively made available for customers, providers, both customers and providers, or other parties. This can be especially beneficial if the customer is traveling but forgot to bring equipment needed for their physical therapy sessions. For example, a customer may use a system enabled by this disclosure to schedule a physical therapy session for their wrist. The customer may realize that they lack the necessary wrist brace with thumb and wrist support. Instead of having to search and locate a medical supply store, the customer may order the desired wrist brace via the interface accessible using the product marketplace module. The ordered product may then be picked up from a supply store, brought to the session by the provider, shipped to the customer, delivered to the customer, or otherwise acquired by the customer. Additional illustrative products that may be offered via the product marketplace module may include braces and straps, knee braces, wrist braces, palm braces, tennis braces, back braces, anatomical models and charts, billable products, aids to daily living, lymphedema, pain relief and lotions, durable medical equipment, exercise equipment, and other products and equipment that will be appreciated by a person of skill in the art having the benefit of this disclosure.

In operation, a method may be provided for coordinating physical therapy, health, and wellness services using an electronically accessible system. Those of skill in the art will appreciate that the following methods are provided to illustrate an embodiment of the disclosure and should not be viewed as limiting the disclosure to only those methods or aspects. Skilled artisans will appreciate additional methods within the scope and spirit of the disclosure for performing the operations provided by the examples below after having the benefit of this disclosure. Such additional methods are intended to be included by this disclosure.

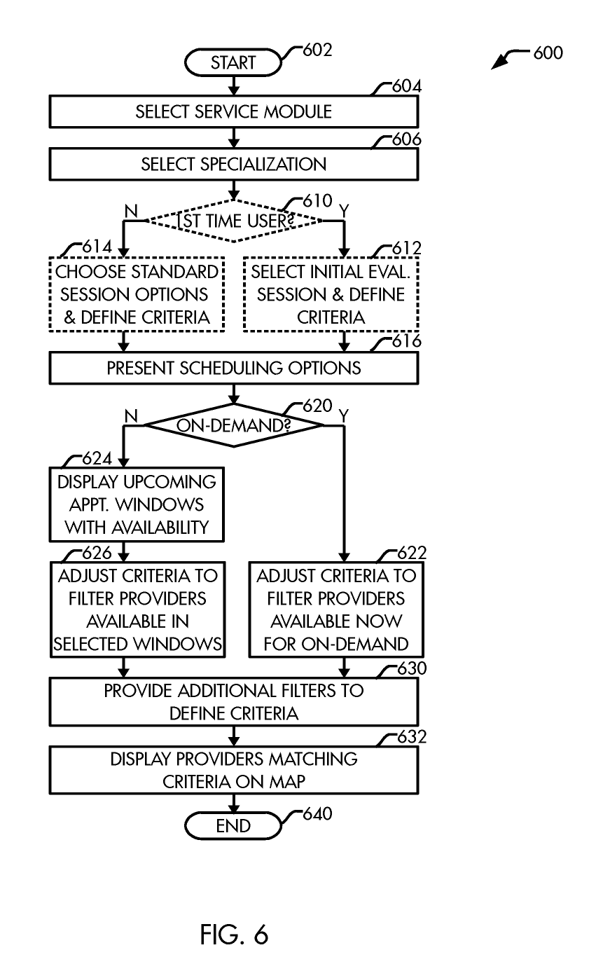

Referring now to flowchart 600 of FIG. 6, an illustrative operation for locating available physical therapy, health, and wellness providers will be described, without limitation. Starting with Block 602, the operation may begin by the customer selecting a service module. (Block 604). As discussed above, a service module may be related to a type of health or wellness service provided by a provider. For example, a service module may be selected relating to massage, personal trainer, physical therapy, or another health and wellness service. The user may then select a specialization provided by the service module, if available. (Block 606). Specializations may further refine the health and wellness services offered by a provider, for example, indicating a specialty for geriatric health and wellness customers.

In an optional step, it may then be determined if the customer is a first-time user. (Block 610). If it is determined at Block 610 that the customer is a first-time user, the customer may be selected to attend an evaluation session. (Block 612). The criteria for health and wellness services to be listed may be defined with consideration of this selection. If it is determined at Block 610 that the customer is not a first-time user, the customer may select standard session options. (Block 614). The criteria for health and wellness services to be listed may be defined with consideration of this selection. After the operations of Block 614 or Block 612 to determine the status of whether a user is a first-time user, or Block 606 if no determination is made whether the user is a first-time user, the customer may then be presented with additional scheduling options. (Block 616).

It may then be determined whether the customer desires on-demand health and wellness services. (Block 620). If it is determined at Block 620 that on-demand services are desired, the criteria may be adjusted to filter for providers available approximately now for providing health and wellness services on-demand. (Block 622). If it is determined at Block 620 that on-demand services are not desired, openings for upcoming session windows with availability may be presented. (Block 624). The customer may indicate acceptable session windows. The criteria may be adjusted to filter for providers available within the selected session windows for providing health and wellness services at the selected time. (Block 626).

After the operation of Block 622 or Block 626, the customer may be presented with the opportunity to select additional conditions to filter and define the criteria. (Block 630). Once the criteria are selected and the search is performed, the customer may view providers matching the criteria on a map. (Block 632). The map with the matching providers may be viewable via an interface, for example, on a customer computerized device. The operation may then end at Block 640.

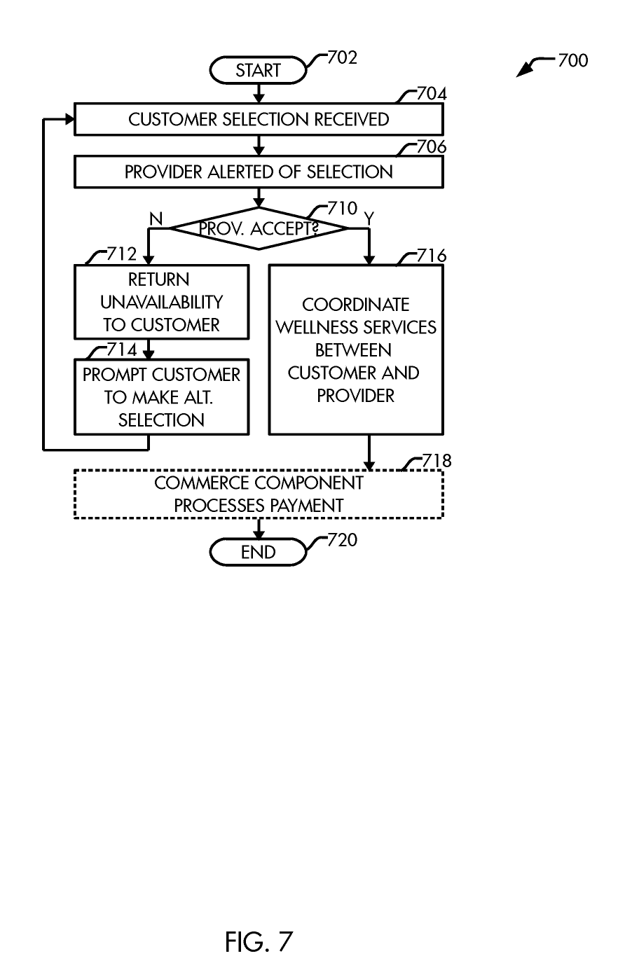

Referring now to flowchart 700 of FIG. 7, an illustrative operation for selecting and scheduling physical therapy, health, and wellness services generally will be described, without limitation. Starting with Block 702, the operation may begin with receipt of a customer selection of a provider. (Block 704). The provider may be alerted that he or she has been selected by the customer. (Block 706).

The provider may be given the opportunity to accept the session. (Block 710). If it is determined at Block 710 that the provider does not accept the session, an indication of unavailability may be returned to the customer. (Block 712). The customer may then be prompted to make an alternative selection. (Block 714). Alternatively, after the service request is posted to substantially all providers within a surrounding vicinity no provider accepts the request, a notification may be sent to the customer estimating an amount of time until such a requested service may be matched. After the selection is made at Block 714, the operation may then return to Block 704, where another customer selection may be received.

If it is determined at Block 710 that the provider accepts the session, the provision of health and wellness services may be coordinated between the provider and the customer. (Block 716). The customer may then be substantially automatically charged for the wellness service. (Block 718). Skilled artisans will appreciate alternative payment structures after having the benefit of this disclosure, for example, manual billing by a provider, customer interaction with the commerce component for payment and checkout, and other payment structures. The operation may then terminate at Block 720.

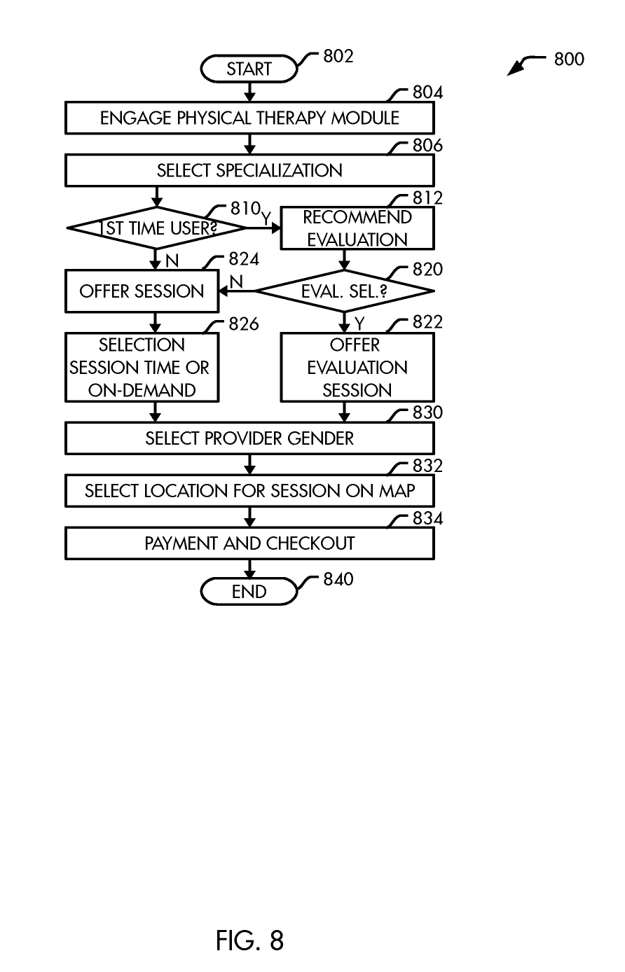

Referring now to flowchart 800 of FIG. 8, an illustrative operation for a physical therapy module will be described, without limitation. Starting with Block 802, the operation may begin by engaging the physical therapy module to locate and coordinate a physical therapy wellness session. (Block 804). A customer interacting with the physical therapy module may initially select a specialization. (Block 806). Specializations may include, for example, geriatrics, pediatrics, sports, orthopedic, neurologic, women's health, or other specialization categories.

It may then optionally be determined if the customer is a first-time user. (Block 810). If it is determined at Block 810 that the customer is a first-time user, an aspect of the interface may be provided to the customer to recommend an initial evaluation. (Block 812). This aspect may be, for example, a pop-up window. It may then be determined whether the customer selects to schedule an evaluation session. (Block 820). If it is determined at Block 820 that the customer wishes to schedule an evaluation session, the customer may be provided with an aspect of the interface offering an evaluation session to be scheduled. (Block 822). The customer may additionally be provided with an option to select to view initial evaluation variables. If the customer engages the initial evaluation variables, an evaluation option may be selected to define the criteria for a session. For example, an initial evaluation session may be selected with a 60-minute duration and $150 costs, without limitation.

If it is determined at Block 810 that the customer is not a first-time user, or if it is determined at Block 820 that the customer does not wish to schedule an evaluation session, the customer may be provided with an interface aspect to define criteria for a session. (Block 824). For example, the customer may be provided with a list of session lengths with corresponding prices to define a duration and cost of the session. After at least part of the criteria is defined for the session, the customer may select whether to schedule a session or seek an on-demand session. (Block 826). The interface may provide an estimated time of arrival for a therapist or other wellness professional if the customer is selecting an on-demand session.

The customer may be provided with additional options to define the criteria for a wellness session, for example, gender of the provider. (Block 830). The customer location may be acquired, which may be displayed to the customer via a map of the interface. (Block 832). The customer may optionally confirm selection. Once the selection is made, the customer may then submit payment and checkout for the wellness contracting service selected. (Block 834). The operation may then terminate at Block 840.

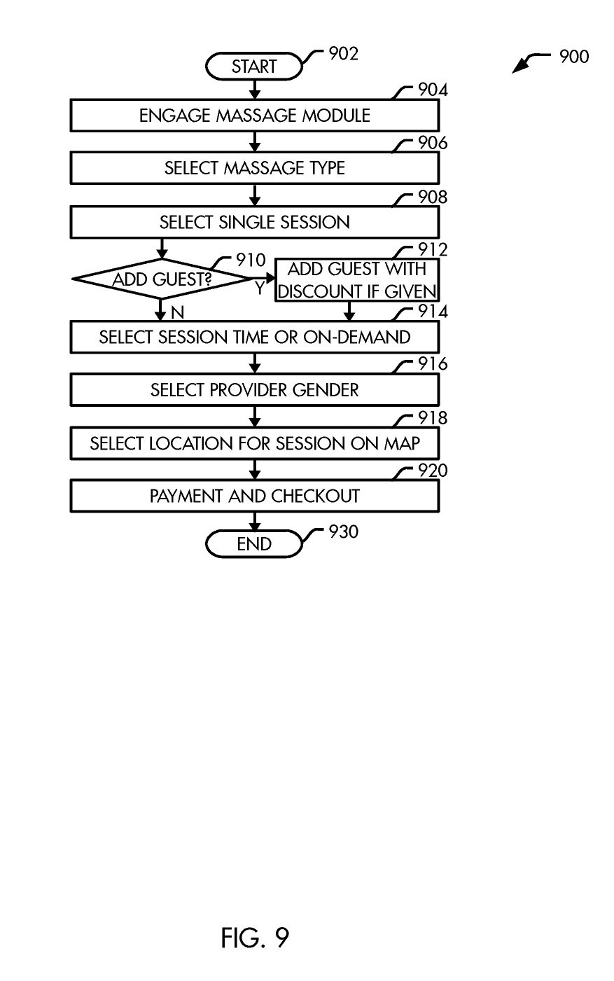

Referring now to flowchart 900 of FIG. 9, an illustrative operation for a massage module will be described, without limitation. Starting with Block 902, the operation may begin by engaging the massage module to locate and coordinate a massage wellness session. (Block 904). A customer interacting with the massage module may initially select a specialization. (Block 906). Specializations may include, for example, Swedish, deep tissue, sports, prenatal, at-work, or other specialization categories.

The customer may then be provided with an interface aspect to define criteria for the session. (Block 908). For example, the customer may select from options of a 60-minute session for $99, 90-minute session for $139, 120-minute session for $169, or virtually any other combination of times and prices that may be determined by a provider, operator, or other party. Optionally, a feature may be included to add a guest to the session. (Block 910). For example, a customer may be provided with the option to add one guest for 15% off, two guests for 20% off, or another number of guests for an incentive or other variation of price that would be apparent to a person of skill in the art after having the benefit of this disclosure. If it is determined at Block 910 that the customer wishes to bring a guest, the customer may be provided an aspect of the interface to give information about the guest and select a price including a discount, if a discount is provided. (Block 912).

After the operation of Block 912, or if the customer decided not to bring a guest at Block 910, the customer may select whether to schedule a session or seek an on-demand session. (Block 914). The interface may provide an estimated time of arrival for a massage therapist or other wellness professional if the customer is selecting an on-demand session.

The customer may be provided with additional options to define the criteria for a wellness session, for example, gender of the provider. (Block 916). The customer location may be acquired, which may be displayed to the customer via a map of the interface. (Block 918). The customer may optionally confirm selection. Once the selection is made, the customer may then submit payment and checkout for the wellness contracting service selected. (Block 920). The operation may then terminate at Block 930.

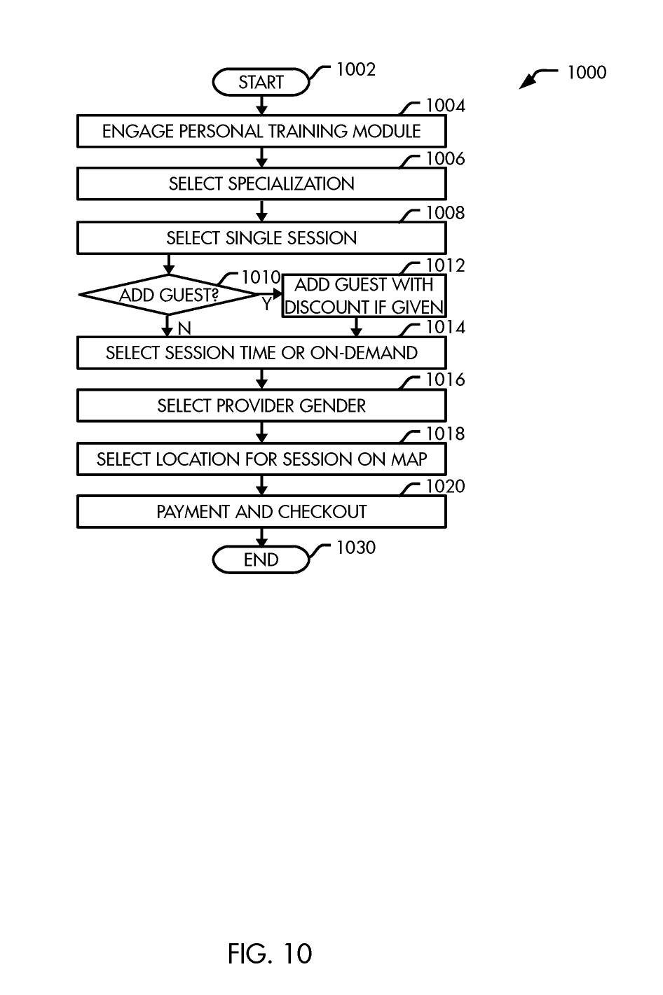

Referring now to flowchart 1000 of FIG. 10, an illustrative operation for a personal training module will be described, without limitation. Starting with Block 1002, the operation may begin by engaging the personal training module to locate and coordinate a personal training wellness session. (Block 1004). A customer interacting with the personal training module may initially select a specialization. (Block 1006). Specializations may include, for example, weight loss, muscle gain, sport specific training, injury rehabilitation, core/flexibility training, endurance training, plyometrics training, explosive performance endurance training, pre/post-natal training, geriatric training, bootcamp group training, or other specialization categories.

The customer may then be provided with an interface aspect to define criteria for the session. (Block 1008). For example, the customer may select from options of a 60-minute session for $99, 90-minute session for $139, 120-minute session for $169, or virtually any other combination of times and prices that may be determined by a provider, operator, or other party. Optionally, a feature may be included to add a guest to the session. (Block 1010). For example, a customer may be provided with the option to add one guest for 15% off, two guests for 20% off, or another number of guests for an incentive or other variation of price that would be apparent to a person of skill in the art after having the benefit of this disclosure. If it is determined at Block 1010 that the customer wishes to bring a guest, the customer may be provided an aspect of the interface to give information about the guest and select a price including a discount, if a discount is provided. (Block 1012).

After the operation of Block 1012, or if the customer decided not to bring a guest at Block 1010, the customer may select whether to schedule a session or seek an on-demand session. (Block 1014). The interface may provide an estimated time of arrival for a massage therapist or other wellness professional if the customer is selecting an on-demand session.

The customer may be provided with additional options to define the criteria for a wellness session, for example, gender of the provider. (Block 1016). The customer location may be acquired, which may be displayed to the customer via a map of the interface. (Block 1018). The customer may optionally confirm selection. Once the selection is made, the customer may then submit payment and checkout for the wellness contracting service selected. (Block 1020). The operation may then terminate at Block 1030.

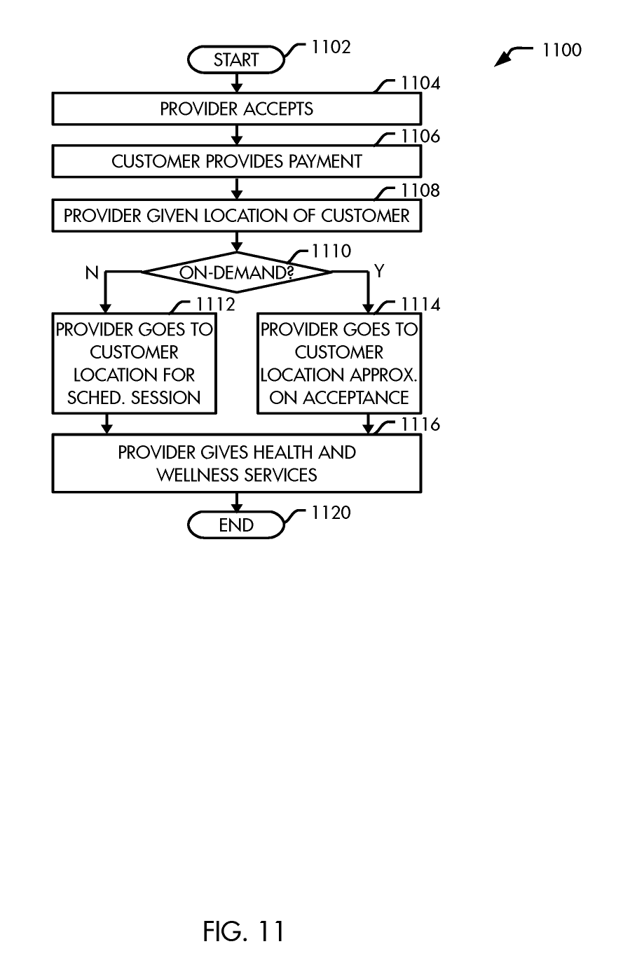

Referring now to flowchart 1100 of FIG. 11, an illustrative operation for a provider accepting a session and providing service will be described, without limitation. Starting with Block 1102, the operation may begin by a provider accepting a session to provide health and wellness services. (Block 1104). The customer may then provide payment for the accepted wellness service session. (Block 1106). After payment is made, the provider may be given the location of the customer. (Block 1108). Alternatively, the customer may designate a location for the session other than the customer's physical location, such as a clinic, gym, or other location.

It may then be determined whether the session was requested to be on-demand. (Block 1110). If it is determined at Block 1110 that the session was not requested to be on-demand, the provider may go to the customer location for a scheduled session. (Block 1112). If it is determined at Block 1110 that the session was requested to be on-demand, the provider may go to the customer location approximately upon acceptance. (Block 1114). Skilled artisans will appreciate that “on-demand” used in the context of this disclosure should not be read as immediately, but as within a reasonably close time. In an alternative example, the customer may also travel to the provider for a scheduled session.

If the session was not requested to be on-demand, the provider may go to the customer location approximately at the scheduled session time. Whether the session was requested as on-demand/or as a scheduled session, the provider may provide the health and wellness services for the customer. (Block 1116). The operation may then terminate at Block 1120.

Referring now to flowchart 1200 of FIG. 12, an illustrative commerce component operation will be described, without limitation. Starting with Block 1202, the operation may begin by receiving a payment request. (Block 1204). The operation may determine whether the service for which payment is requested is eligible for payment by insurance, such as by determining a level of insurance compatibility. (Block 1210). For example, the system may determine if a physical therapist or other provider is administering a session that includes services eligible for billing to insurance that accepts an insurance carried by a customer, such as physical therapy or insurance-eligible massage therapy, without limitation. If it is determined at Block 1210 that the requested service is eligible for payment by insurance, it may be determined whether the insurance carried by the customer is accepted by the requested provider. (Block 1220).

If it is determined at Block 1210 that the requested service is not eligible for payment by insurance, the total fee for the service may be required to be paid by the customer. Additionally, if it is determined at Block 1220 that the requested service is eligible for insurance, but the insurance carried by the customer is unacceptable, the total fee for the service may be required to be paid by the customer.

If it is determined at Block 1210 that the service is eligible for at least partial payment by insurance, and at Block 1220 that the insurance carried by the customer is acceptable, the portion of the wellness service billable to the insurance carrier may optionally be calculated. (Block 1222). The insurance carrier may optionally be invoiced for the billable amount. In one example, the insurance may be substantially automatically invoiced by the illustrative system. In another example, information about the customer and/or the session may be collected to assist the provider with submitting insurance documentation external to the illustrative system. It may also be determined whether any additional fee will be required beyond that covered by insurance. (Block 1224). In alternative embodiments, billing to an insurance company may be performed by the provider outside of the system.

After the operation of Block 1224, or if Block 1210 or Block 1220 are decided in the negative, the operation may determine whether a transaction fee for the requested health and wellness services is required to be paid by the customer. The transaction fee may be included with the portion of the fee for the service to be paid by the customer, if any. (Block 1226). In some embodiments, a transaction fee may be required by a customer even if substantially all the fee for the wellness service is payable by insurance. The payment from the customer of the fee for the wellness service and/or transaction fees may then be processed. (Block 1228). The operation may then terminate at Block 1230.

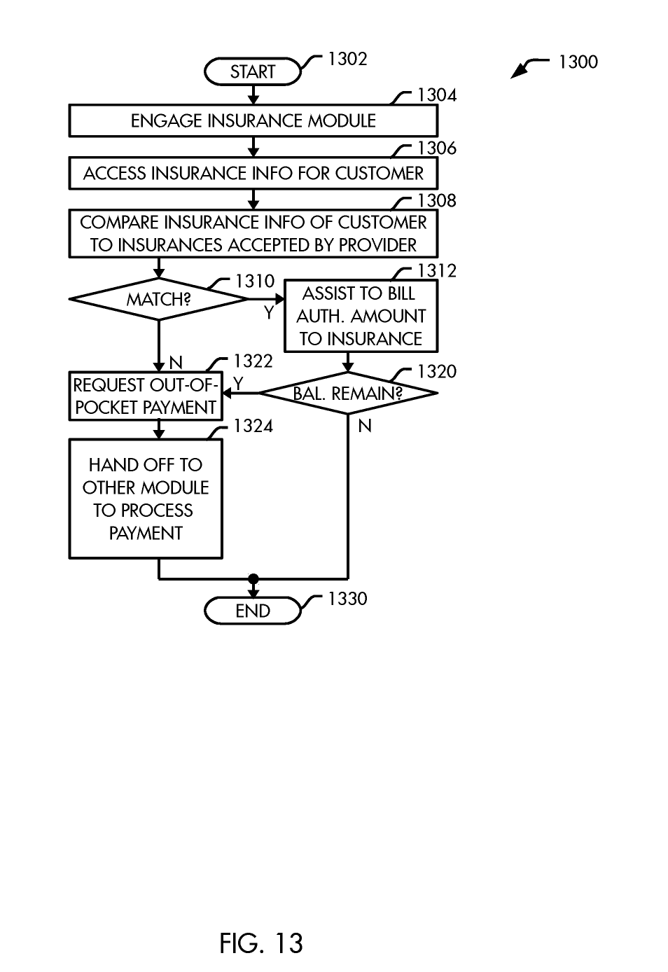

Referring now to flowchart 1300 of FIG. 13, an alternative embodiment of an illustrative insurance module operation will be described, without limitation. Starting with Block 1302, the operation may begin by engaging the insurance module (Block 1304). The operation may then access insurance information for the customer (Block 1306). The accessed insurance information may be included by the user profile for the customer, which may be stored in and/or accessed from a connected database. The operation may compare the insurance information associated with the customer with insurance policies and/or companies accepted by a provider. (Block 1308). In one example, the operation of Block 1308 may determine a level of insurance compatibility.

It may then be determined at Block 1310 whether the customer insurance is within an acceptable level of insurance compatibility and/or matches with an insurance accepted by the provider. If it is determined at Block 1310 that the insurance is within an acceptable level of insurance compatibility and/or matches, the operation may authorize an amount to be billed to the insurance. (Block 1312). In one example, the insurance may be substantially automatically invoiced by the illustrative system. In another example, information about the customer and/or the session may be collected to assist the provider with submitting insurance documentation external to the illustrative system. The operation may then determine whether the authorized amount to bill is sufficient to cover the cost of the service from the provider. (Block 1320). If it is determined at Block 1320 that no additional payment is required, for example, because the insurance covers the total cost, the operation may terminate at Block 1330.

If it is determined at Block 1310 that no match exists between the customer insurance and the insurance accepted by the provider, or if it is determined at Block 1320 that a balance remains after subtracting the amount authorized by the customer insurance, such as an insurance payment deficiency, the operation may request payment of any open balance, for example, out-of-pocket. (Block 1322). To process the payment, the operation may hand off the payment steps to another module, for example, a payment module or third-party payment gateway. (Block 1324). The operation may then terminate at Block 1330.

Referring now to flowchart 1400 of FIG. 14, an illustrative operation for a rebooking module will be described, without limitation. Starting with block 1402, the operation may begin by providing a customer with a view of previous contracted health and wellness services and/or providers. (Block 1404). The customer may view their order history, for example, to select a past order for health and wellness services to view. (Block 1406). The customer may then select to rebook a wellness service with the same provider. (Block 1408). For example, a customer may select to rebook a follow-up personal training wellness session with a personal trainer that the customer liked. Once the customer has selected the desired provider, the customer may confirm the rebooking. (Block 1410).

While selecting a service to request using the rebooking module, the customer may edit variables relating to the session. (Block 1412). For example, a customer may edit session length, session date, and/or other variables. Alternatively, a customer may select to rebook a follow-up session as an on-demand session. (Block 1414). The provider may be given a notification of the rebooking request. An accepted rebooking request may be provided to the selected provider on a map via the interface. (Block 1416). The customer's location may be prepopulated on the map using the location of the last session with the provider, which may be edited by the customer. Payment and checkout for the rebooked session may be conducted as discussed above. (Block 1418). The operation may then terminate at Block 1420.

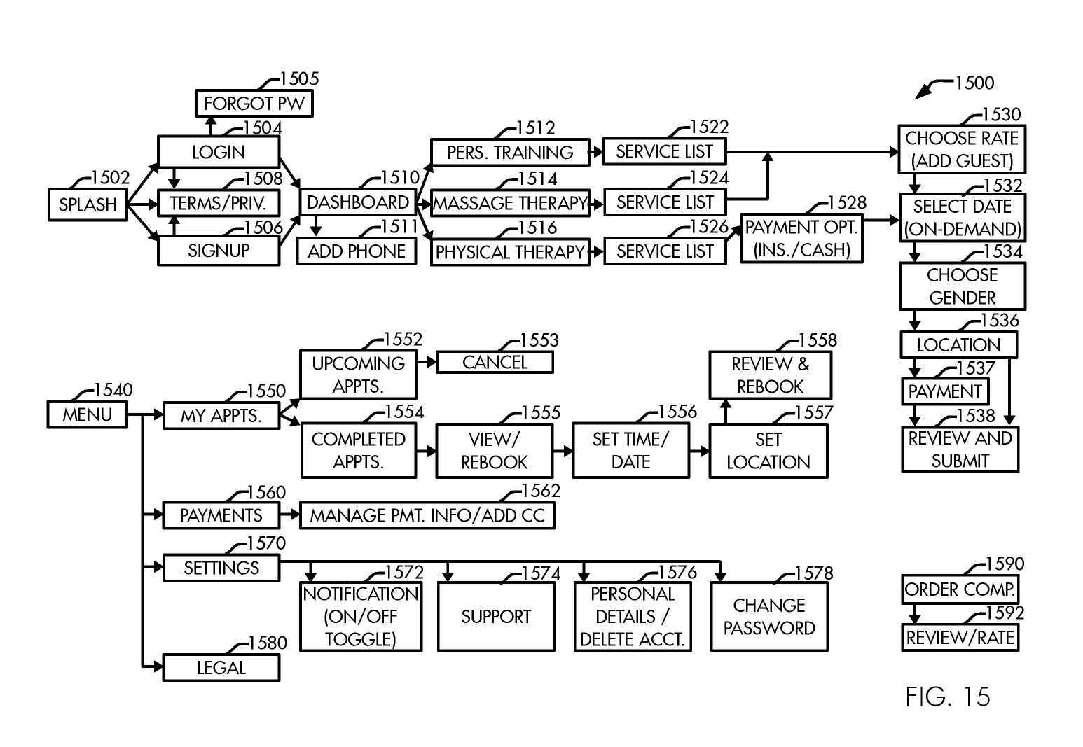

Referring now to flowchart 1500 of FIG. 15, an illustrative operation of an interface usable by a customer will be described, without limitation. Upon accessing the interface, the customer may be provided with the splash screen. (Block 1502). The splash screen may display a logo for the interface, such as a logo of the operator, a skinned logo, a “whitelabel” logo, or another logo.

The customer may be provided with options to login (Block 1504), sign up for an account (Block 1506), or view terms of service and privacy policy information (Block 1508). A person of skill in the art will appreciate additional options that may further be provided by the interface. If a customer has already created an account, but forgot his or her password, the customer may access a forgot password link. (Block 1505).

After a customer has signed up for an account and/or has logged into his or her account, the customer may be directed to a dashboard. (Block 1510). The customer may optionally provide a phone number through the dashboard. (Block 1511).

The customer may then choose a health and wellness service to request. In this example, the customer may choose between health and wellness services that may include personal training (Block 1512), massage therapy (Block 1514), and physical therapy (Block 1516). Those of skill the art will appreciate additional health and wellness services that may be provided by the system of this disclosure and listed through this interface.

If a customer chooses to request a personal training service at Block 1512, the customer may be provided with a service list that includes multiple descriptions of personal training subset services. (Block 1522). Similarly, if the customer requests a massage therapy service at Block 1514, the customer may be provided with the service list describing multiple massage therapy subset services. (Block 1524). If the customer requests a physical therapy service at Block 1516, the customer may be provided a service list with descriptions of physical therapy subset services available. (Block 1526). For physical therapy and other health and wellness services that may include billing to an insurance provider, the customer may be provided with an additional aspect of the interface to determine a payment option. Payment options may include insurance, cash, and other payment options. (Block 1528).

If the customer has selected personal training at Block 1512, massage therapy at Block 1514, or another health and wellness service through which payment may be collected by the system, the customer may choose a rate. (Block 1530). The customer may also select whether to add a guest, which may optionally be provided a discounted rate. The customer may then choose a date for the service or indicated desire for on demand service. (Block 1532). If the customer chooses the physical therapy, or another health or wellness service that is billed externally, the customer may select the date for the service or indicate a desire for on demand service.

Once the timing of the service has been selected by the customer, the customer may optionally choose the gender of a service provider. (Block 1534). The gender selection by the customer may add a note to the provider to facilitate meeting this request.

The location of the customer may then be determined to facilitate meeting the provider of the health and wellness service. (Block 1536). If the requested service will be billed through the system, payment information may be provided by the customer. (Block 1537). In one example, payment information may be stored so that future requests for health and wellness services do not require an additional entry of credit card or other payment information. Once information to request a health and wellness service has been acquired, the customer may review and confirm the details of the request. The customer may then submit the request. (Block 1538).

The customer interface may additionally include a menu. (Block 1540). The menu may include navigation features to view a customer's sessions (Block 1550), payments (Block 1560), settings (Block 1570), legal (Block 1580), and/or other aspects. The customer interface may additionally include an order complete interface. (Block 1590). The customer may leave feedback, reviews, and/or ratings through an aspect of the order complete interface. (Block 1592).

If viewing the customer sessions through Block 1550, the customer may see upcoming sessions scheduled with a health and wellness service provider. (Block 1552). The customer may optionally cancel upcoming sessions. (Block 1553). A customer may additionally view completed sessions with health and wellness service providers. (Block 1554). The customer may optionally rebook sessions with prior service providers. (Block 1555). If rebooking, the customer may set a date and time for the future session (Block 1556), location of the session (Block 1557), review, and book the requested session. (Block 1558).

If viewing the payments option through Block 1560, the customer may manage the payment methods available to request health and wellness services. (Block 1562). For example, a customer may add and/or edit credit card information. The customer may optionally enter health insurance provider information to facilitate with matching of health and wellness service providers that accept a customer's insurance.

Through the settings menu of Block 1570, the customer may access settings to control the customer account and/or interaction with the interface. For example, the customer may toggle notifications on or off (Block 1572), request support (Block 1574), view personal details and/or delete their customer account (Block 1576), change password (Block 1578), and/or otherwise change settings for the customer.

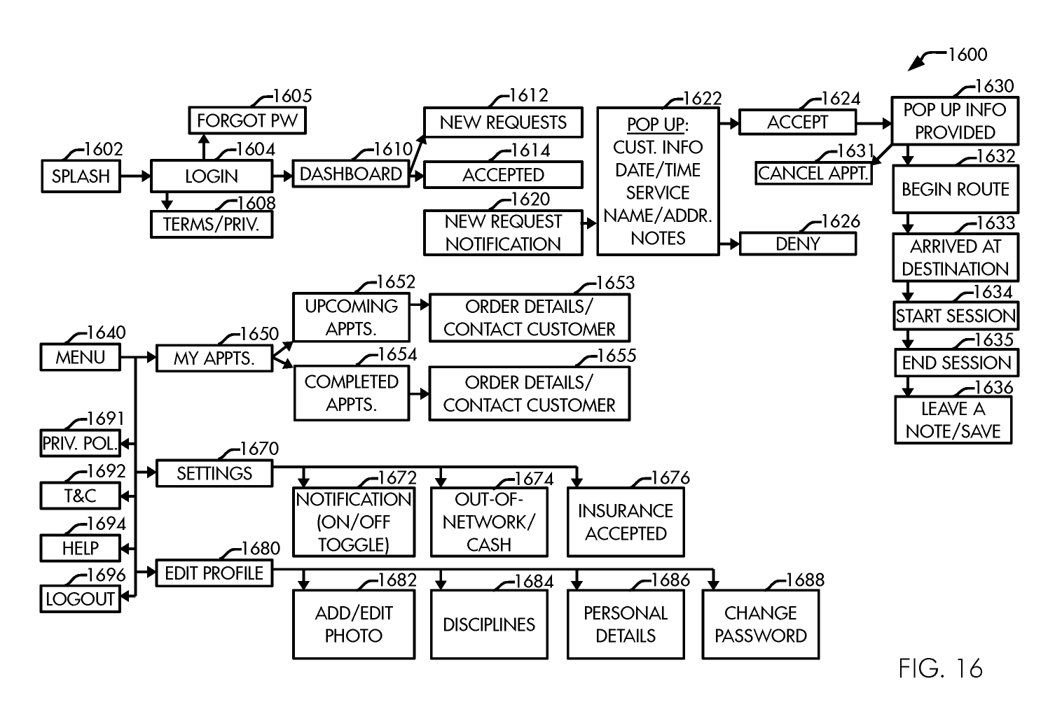

Referring now to flowchart 1600 of FIG. 16, an illustrative operation of an interface usable by a provider will be described, without limitation. Upon accessing the interface, a provider may be displayed a splash screen. (Block 1602). The splash screen may display a logo for the interface, such as a logo of the operator, a skinned logo, a “whitelabel” logo, or another logo. The provider may be given an option to login to the system. (Block 1604). If the provider forgot their password, they may be given an option to recover the credential information. (Block 1605). The provider may also be given access to terms of service, privacy policy, and other administrative documents. (Block 1608).

After logging in at Block 1604, the provider may be directed to a dashboard. (Block 1610). From the dashboard, the provider may manage his or her requests. For example, the provider may view new request available for acceptance. (Block 1612). Additionally, a provider may view requests he or she has already accepted. (Block 1614).

The provider may be notified of a new service request by a customer interacting with a customer interface of the system. (Block 1620). The new request notification may be displayed to the provider as a pop up and/or another interface aspect. (Block 1622). The notification request may include information such as the date and time of the requested service, the type of service, the user's name and address, notes about the service, and other information that would facilitate a provider giving the service. At least part of this information may be provided by the user.

The provider may choose to deny the request. (Block 1626). If the provider denies the request, the notification may be removed from the providers interface. The provider may also choose to accept the request. (Block 1624). If the provider accepts the request, information about the service may be delivered to the provider to confirm the service request. (Block 1630).

The provider may be given an opportunity to cancel the session. (Block 1631). If the session is cancelled, the notification may be removed from the provider interface. Alternatively, the provider may choose to begin a route to the health and wellness service session. (Block 1632). The provider may interact with the interface to indicate that the provider has begun the route to the customer. Once the provider arrives at the destination for the session, the provider may interact with the interface to indicate arrival. (Block 1633). Once the provider and the customer meet in person, the provider may start the health and wellness service session. (Block 1634). After the health and wellness service session has completed, the provider may end the session, for example, by interacting with an aspect of the interface. (Block 1635). The provider may then leave a note about the session, customer, and other details to be saved with the providers profile. (Block 1636).

The provider interface may include a menu (Block 1640), which may give the provider access to information regarding a provider's sessions (Block 1650), settings (Block 1670), profile information (Block 1680), privacy policy (Block 1691), terms and conditions (Block 1692), help (Block 1694), and ability to log out of the system (Block 1696). If the provider selects the sessions option at Block 1650, the provider may view upcoming sessions (Block 1652) and/or completed sessions (Block 1654). From the upcoming sessions view of Block 1652, the provider may view details about upcoming sessions, contact the customer for an upcoming session, cancel an upcoming session, and otherwise view or modify details about upcoming sessions. (Block 1653). From the completed sessions view of Block 1654, the provider may additionally view details and/or notes about completed sessions with customers. (Block 1655).

From the settings menu of Block 1670, a provider may modify settings for the account and/or rendering of health and wellness services. For example, the provider may select whether to receive notifications for customer request for health and wellness services. (Block 1672). The provider may additionally select whether to receive payment for health and wellness services through the system, outside of the network, via cash, or otherwise. (Block 1674). Through the settings, the provider may indicate whether health insurance is accepted and, if so, through which carriers. (Block 1676).

From the profile menu of Block 1680, the provider may additionally edit his or her profile. For example, the provider may add or edit a photo (Block 1682), specify disciplines (Block 1684), add or modify personal details (Block 1686), change his or her password (Block 1688), and otherwise edit the profile. Disciplines may include specializations and focus areas of practice. The number of disciplines that may be listed by a provider can be based on a tier of membership payment.

While various aspects have been described in the above disclosure, the description of this disclosure is intended to illustrate and not limit the scope of the invention. The invention is defined by the scope of the appended claims and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the following claims.

What is claimed is:

1. A system for improving provider and customer matching comprising:
a dashboard component accessed using a network-connected device with a display, the dashboard component comprising:
a customer module manipulable by the customer via the network-connected device to at least view provider information and request a session with the provider,
a provider module manipulable by the provider via the network-connected device to at least view the session that is requested by the customer and selectively accept the session, and
a mapping module manipulable by the customer to set a location for the session and manipulable by the provider to receive the location;
a service coordination component to improve sharing of information about the session between the customer and the provider, the service coordination component comprising:
a matching module to associate the customer and the provider to coordinate the session, the matching module applying criteria defined by the customer to filter a list of providers according to a rule, the provider included in the list of providers that is compliant with the rule being presented to the customer for selection to administer the session, and
an on-demand module to facilitate expedited scheduling of the session chronologically close to a time that the session is requested by the customer; and
a commerce component to exchange a value from the customer requesting the session to the provider administering the session;
wherein the customer is associated with customer information that is stored in a database;
wherein the provider is associated with the provider information that is stored in the database;
wherein the customer information and the provider information stored by the database is accessible via the network-connected device.

2. The system of claim 1, wherein the commerce component comprises:
a payment module to receive at least part of the value from the customer, validate the exchange of the value that is received, and deliver the value that is validated to the provider.

3. The system of claim 2, wherein the commerce component further comprises:
an insurance module to compare the session that is requested, an insurance policy of the customer, and a list of acceptable insurance of the provider to determine a level of insurance compatibility; and
wherein if the level of insurance compatibility is sufficient to at least partially pay for the session, the insurance module assists with invoicing the insurance policy for the session.

4. The system of claim 3, wherein if the level of insurance compatibility is insufficient to fully pay for the session, the insurance module reports an insurance payment deficiency to the payment module to assist with the exchange of the value defined by the insurance payment deficiency by the customer.

5. The system of claim 2, wherein the commerce component further comprises:
a product marketplace module to sell goods; and
wherein the exchange of the value for the goods that are sold is assisted by the payment module.

6. The system of claim 1, wherein the dashboard component further comprises:
an onboarding module to receive the customer information for the customer desiring to search for the provider for the session and store the customer information in the database, and to receive the provider information for the provider desiring to administer the session to the customer and store the provider information in the database.

7. The system of claim 1, wherein the dashboard component comprises:
an administrator module to at least partially adjust the criteria and to access performance data.

8. The system of claim 1, wherein the commerce component comprises:
a monetization module to determine and collect a subscription fee from a whitelabel provider.

9. A system for improving provider and customer matching comprising:
a dashboard component accessed using a network-connected device with a display, the dashboard component comprising:
a customer module manipulable by the customer via the network-connected device to at least view provider information and request a session with the provider, and
a provider module manipulable by the provider via the network-connected device to at least view the session that is requested by the customer and selectively accept the session;
a service coordination component to improve sharing of information about the session between the customer and the provider, the service coordination component comprising:
a matching module to associate the customer and the provider to coordinate the session, the matching module applying criteria to filter a list of providers according to a rule, the provider included in the list of providers that is compliant with the rule being presented to the customer for selection to administer the session, and
an on-demand module to facilitate expedited scheduling of the session chronologically close to a time that the session is requested by the customer; and
a commerce component to exchange a value from the customer requesting the session to the provider administering the session, the commerce component comprising:
a payment module to receive at least part of the value from the customer, validate the exchange of the value that is received, and deliver the value that is validated to the provider,
an insurance module to compare the session that is requested, an insurance policy of the customer, and a list of acceptable insurance of the provider to determine a level of insurance compatibility,
wherein if the level of insurance compatibility is sufficient to at least partially pay for the session, the insurance module assists with invoicing the insurance policy for the session, and
wherein if the level of insurance compatibility is insufficient to fully pay for the session, the insurance module reports an insurance payment deficiency to the payment module to assist with the exchange of the value defined by the insurance payment deficiency by the customer.

10. The system of claim 9, wherein the dashboard component further comprises:
a mapping module manipulable by the customer to set a location for the session and manipulable by the provider to receive the location;
wherein the customer is associated with customer information that is stored in a database;
wherein the provider is associated with the provider information that is stored in the database; and
wherein the customer information and the provider information stored by the database is accessible via the network-connected device.

11. The system of claim 10, wherein the dashboard component further comprises:
an onboarding module to receive the customer information for the customer desiring to search for the provider for the session and store the customer information in the database, and to receive the provider information for the provider desiring to administer the session to the customer and store the provider information in the database.

12. The system of claim 9, wherein the commerce component further comprises:
a product marketplace module to sell goods; and
wherein the exchange of the value for the goods that are sold is assisted by the payment module.

13. The system of claim 9, wherein the dashboard component comprises:
an administrator module to at least partially adjust the criteria and to access performance data.

14. The system of claim 9, wherein the commerce component comprises:
a monetization module to determine and collect a subscription fee from a whitelabel provider.

15. A method for improving provider and customer matching comprising:
(a) viewing provider information and requesting a session by the customer via manipulating a customer module of a dashboard component via a network-connected device with a display;
(b) viewing the session that is requested by the customer and selectively accepting the session by the provider via manipulating a provider module of the dashboard component via the network-connected device;
(c) associating the customer and the provider via a matching module of a service coordinating component to coordinate the session, further comprising:
(i) applying criteria to filter a list of providers according to a rule, and
(ii) presenting to the customer the provider included in the list of providers that is compliant with the rule for selection to administer the session,
(d) defining a location for the session, further comprising:
(i) setting the location by the customer via a mapping component, and
(ii) receiving the location by the provider via the mapping component;
(e) exchanging value from the customer requesting the session to the provider administering the session via a commerce component, further comprising:
(i) receiving at least part of the value from the customer via a payment module,
(ii) validating an exchange of the value that is received, and
(iii) delivering the value that is validated to the provider; and
(f) comparing the session that is requested, an insurance policy of the customer, and a list of acceptable insurance of the provider to determine a level of insurance compatibility via an insurance module, further comprising:
(i) assisting with invoicing the insurance policy for the session if the level of insurance compatibility is sufficient to at least partially pay for the session, and
(ii) reporting an insurance payment deficiency to the payment module to assist with the exchange of the value defined by the insurance payment deficiency by the customer if the level of insurance compatibility is insufficient to fully pay for the session.

16. The method of claim 15, further comprising after step (b):
(g) scheduling the session chronologically close to a time that the session is requested by the customer via an on-demand module of the service coordination component;
wherein the customer is associated with customer information that is stored in a database;
wherein the provider is associated with the provider information that is stored in the database; and
wherein the customer information and the provider information stored by the database is accessible via the network-connected device.

17. The method of claim 16, further comprising:
(h) receiving the customer information via an onboarding module for the customer desiring to search for the provider for the session and storing the customer information in the database, and
(j) receiving the provider information via the onboarding module for the provider desiring to administer the session to the customer and storing the provider information in the database.

18. The method of claim 15, further comprising:
(k) selling goods via a product marketplace module of the commerce component; and
wherein the exchange of the value for the goods that are sold is assisted by the payment module.

19. The method of claim 15, further comprising:
(l) at least partially adjusting the criteria and accessing performance data via an administrator module of the dashboard component.

20. The method of claim 15, further comprising:
(m) determining and collecting a subscription fee from a whitelabel provider via a monetization module of the commerce component.
